SEQUENCE LISTING (7 sequences)

SEQ ID NO: 1 - protein, 158 aa, Glycine max
MARVKHTPASRKSAKKQAPRASTSTQPPPQSQSPATRERRRAQQVEPQQEPEAQGRKKRRNRSGTVALREIRHFQRSCELLIPAAPFIRCVKQITNQFSTEVSRWTPEAVVALQEAAEEYLVHLFEDGMLCAIHARRITLMKKDIELARRLGGIGRPW

SEQ ID NO: 2 - DNA, 964 bp, Glycine max
aaaccgcttcattttgaaaattcacaaaccaatagtgaaaagagacagagagggaaaaagaagaagaaaagaaaaagaaaattgcaaaaagcgaaaccgaaaaccaaatccgaagagtgaaggttcgttgaatcccatggcgagagtgaagcacacgccagcttctcgcaaatccgctaaaaagcaagcgccacgcgcatccacttccacgcagccgccaccacaatcccaatcgcctgcaactagagagaggaggagagctcaacaagtggagccgcagcaggagcctgaggctcagggaaggaagaagaggcgcaataggtcgggaactgtggcgcttcgtgagattcggcattttcagaggagttgtgagcttctcatcccggctgctccctttatcagatgtgtcaaacagattacaaatcaattctctacggaggtgtctcgttggacacctgaagctgtggtagcacttcaggaggcagctgaggaatatctggttcacttatttgaagatggaatgctctgtgcaattcatgcaaggcgtattactcttatgaaaaaggacattgagttggcccggaggcttggaggaataggaaggccttggtgagtggacgatactagtagaagaccattggttcttttgtttgtaaaagttcacacagtagggggatttcaatgaagggaggcattggtgcttctgtactccgcactctaattgcagtgattatttgatattaaatgttaggaactagacactaactaatgtatgccaatggttatatctctgtaggtgaaacaaccattatagaatggttgctactataactttaaattgatgagtgaggtaatagtgtaacattgtagcaatgaagtattctaagattattgtcattcatgttgattttctgtggtagaaggaattcaagggtccacaaaatgtcaaggcttaaaattttcc

SEQ ID NO: 3 - protein, 43 aa, Arabidopsis thaliana
MARTKQSARKSHGGKAPTKQLATKAARKSAPTTGGVKKPHRFR

SEQ ID NO: 4 - protein, 96 aa, Glycine max
PGTVALREIRHFQRSCELLIPAAPFIRCVKQITNQFSTEVSRWTPEAVVALQEAAEEYLVHLFEDGMLCAIHARRITLMKKDIELARRLGGIGRPW

SEQ ID NO: 5 - protein, 139 aa, Artificial Sequence
  Fusion protein of Arabidopsis H3 N-terminal tail and soybean CenH3 fold
MARTKQSARKSHGGKAPTKQLATKAARKSAPTTGGVKKPHRFRPGTVALREIRHFQRSCELLIPAAPFIRCVKQITNQFSTEVSRWTPEAVVALQEAAEEYLVHLFEDGMLCAIHARRITLMKKDIELARRLGGIGRPW

SEQ ID NO: 6 - DNA, 435 bp, Artificial Sequence
  Synthetic DNA encoding the Arabidopsis H3 N-terminal tail fused to the CenH3 histone fold of Glycine Max
accaccatggctcgtacgaagcaatccgcgagaaaatcacacggaggaaaagctccgacgaagcagctcgctaccaaggcggcaaggaaatctgcaccgactaccggaggagtcaagaaacctcaccgtttccgtccgggcaccgtggccctccgggagatccggcacttccagaggagctgcgagctcctgatccccgcagcaccgttcatcaggtgcgtgaagcagatcaccaaccagttcagcactgaggtcagccggtggaccccagaggccgtcgtcgctctccaggaggcggcagaggagtatctcgtccacctgttcgaggacggcatgctctgcgccatccacgccaggcgcatcaccctcatgaagaaggacatcgagctcgcgcgcagactcggaggcatcggcagaccgtggtgatgaacacac

SEQ ID NO: 7 - protein, 139 aa, Artificial Sequence; identical to SEQ ID NO: 5
  Synthetic Construct

(19) United States
(12) Patent Application Publication
(10) Pub. No.: US 2016/0192608 A1
(43) Pub. Date: Jul. 7, 2016

(54) Epigenetically Enhanced Double Haploids

(71) Applicant: The Board of Regents of the University of Nebraska, Lincoln, NE (US)

(72) Inventors: Michael E. Fromm, Lincoln, NE (US); Sally A. Mackenzie, Lincoln, NE (US)

(21) Appl. No.: 14/599,870

(22) Filed: Jan. 19, 2015

Related U.S. Application Data
(60) Provisional application No. 61/930,602, filed on Jan. 23, 2014.
(60) Provisional application No. 62/041,227, filed on Aug. 25, 2014.

(51) Int. Cl. A01H 1/08

(52) U.S. Cl. CPC A01H 1/08

(57) ABSTRACT

The present invention provides methods for obtaining plants that exhibit useful traits by producing double haploids of plants with epigenetic modifications initiated in a parental line by suppression of MSH1. In addition, plants that exhibit the useful traits, parts of the plants including seeds, and products of the plants are provided.

### CROSS-REFERENCE TO RELATED APPLICATIONS

This application claims the benefit of U.S. Provisional Patent Application No. 61/930,602 filed Jan. 23, 2014, and U.S. Provisional Patent Application No. 62/041,227 filed Aug. 25, 2014, which are each incorporated hereinby reference in their entireties.

### INCORPORATION OF SEQUENCE LISTING

The sequence listing contained in the file named “PatentIN_SEQ FILES_1_19_2015.txt”, which is 13,036 bytes in size (measured in operating system MS-Windows), contains 6 sequences, and which was created on Jan. 19, 2015, is contemporaneously filed with this specification by electronic submission (using the United States Patent Office EFS-Web filing system) and is incorporated herein by reference in its entirety.

### STATEMENT REGARDING FEDERALLY SPONSORED RESEARCH OR DEVELOPMENT

None.

### BACKGROUND

The use of doubled-haploid plants as a technique for plant breeding is well established and has become a routine practice for breeders of most crops, including those of canola, wheat, barley, and maize. The main advantage of generating doubled-haploid plants is the greatly reduced time required to achieve genetic homozygosity. Double haploids are produced by the doubling of a haploid set of chromosomes (1N) derived from a heterozygous diploid parent plant (2N) to produce a genetically homozygous plant (2N). Alternatively, tetraploid crops (4N) can be reduced to ‘haploid’ (2N) levels and made into double haploids (4N) from the haploid (2N). Similarly, hexaploid plants can be haploid (3N) or double haploids (6N).

### Doubling Chromosome Number of Haploid Plants to Make Diploid Plants.

Haploid plants that comprise only a single set of chromosomes are infertile and must be doubled in their chromosome complement before use in breeding. Techniques for doubling the chromosome number in haploid plants using colchicine and other chemicals that disturb the cytoskeleton of cells are well known in the literature. Double haploids are produced by the doubling of a set of chromosomes from a haploid plant (1N) to produce a diploid plant (2N). For example, see, Wan, et al., “Efficient Production of Doubled Haploid Plants Through Colchicine Treatment of Anther-Derived Maize Callus,” Theoretical and Applied Genetics, 77:889-892 (1989), U.S. Pat. No. 7,135,615, incorporated herein by reference in its entirety, Hays et al. In: Doubled Haploid Production in Crop Plants, Maluszynski ed. (Dordrecht: Kluwer Academic Publishers) Ch. 2.1, pp. 5-14 (2003), and Dunwell 2010 Plant Biotechnol J. May 1; 8(4):377-424.

### General Transgenic Methods of Making Haploid Plants.

U.S. Pat. No. 8,618,354, incorporated herein by reference in its entirety, describes a method wherein alteration of the plant's CenH3 leads to haploid plants, which can then be colchicine treated to form double haploids. This CenH3 transgenic method provides a general method of haploid production for all plants. U.S. Pat. No. 8,242,327, incorporated herein by reference in its entirety, describes a second transgenic method for producing non-recombined haploid plants, which can then be treated to form double haploids.

### Non-Transgenic Methods of Making Haploid Plants.

Methods for obtaining haploid plants are also disclosed in Kobayashi, M., et al., Journ. of Heredity, 71(1):9-14 (1980), Pollacsek, M., 12(3):247-251, Agronomie, Paris (1992); Cho-Un-Haing, et al., Journ. of Plant Biol., 39(3):185-188 (1996); Verdoodt, L., et al., 96(2):294-300 (February 1998); Genetic Manipulation in Plant Breeding, Proceedings International Symposium Organized by EUCARPIA, Berlin, Germany (Sep. 8-13, 1985); Thomas, W J K, et al., “Doubled haploids in breeding,” in Doubled Haploid Production in Crop Plants, Maluszynski, M., et al. (Eds.), Dordrecht, The Netherland Kluwer Academic Publishers, pp. 337-349 (2003).

Microspore culture for haploid production is possible for many plant species, including but not limited to plants in the families Graminae, Leguminoceae, Cruciferaceae, Solanaceac, Cucurbitaceae, Rosaccae, Poaceae, Lilaceae, Rutaceae, Vitaceae, including such species as corn (Zea mays), wheat (Triticum aestivum), rice (Oryza sativa), oats, barley, canola (Brassica napus, Brassica rapa, Brassica oleracea, and Brassica juncea), cotton (Gossypium hirsuitum L.). various legume species (e.g., soybean [Glycine max], pea [Pisum sativum], etc.), grapes [Vitis vinifera], and a host of other important crop plants. Microspore embryogenesis, both from anther and microspore culture, has been described in more than 170 species, belonging to 68 genera and 28 families of dicotyledons and monocotyledons (Raghavan, Embryogenesis in Agniosperms: A Developmental and Experimental Study, Cambridge University Press, Cambridge, England, 1986; Rhagavan, Cell Differentiation 21:213-226, 1987; Raemakers et al., Euphytica 81:93-107, 1995). For a detailed discussion of microspore isolation, culture, and regeneration of double haploid plants from microspore-derived embryos [MDE] in Brassica napus L., see Nehlin, The Use of Rapeseed (Brassica napus L.) Microspores as a Tool for Biotechnological Applications, doctoral thesis, Swedish University of Agricultural Sciences, Uppsala, Sweden, 1999; also Nehlin et al., Plant Sci. 111:219-227, 1995, and Nehlin et al., Plant Sci. 111:219-227, 1995).

Various methodologies of making double haploid plants in wheat have been developed (Laurie, D. A. et al., Plant Breeding, 1991, 106:182-189: Singh, N. et al., Cereal Research Communications, 2001, 29:289-296; Redha, A. et al., Plant Cell Tissue and Organ Culture, 2000, 63:167-172; U.S. Pat. No. 6,362,393). Haploid induction systems have been developed for various plants to produce haploid tissues, plants and seeds. The haploid induction system can produce haploid plants from any genotype by crossing a selected line (as female) with an inducer line. Such inducer lines for maize include Stock 6 (Coe, 1959, Am. Nat. 93:381-382; Sharkar et al., 1966, Genetics 54:453-464), KEMS (Deimling et al., 1997, Vortr. Pflanzenzuchtg 38:203-224), or KMS and ZMS (Chalyk et al., 1994, MNL 68:47; Chalyk et al., 2000, Plant Breeding 119:363-364), and indeterminate gametophyte (ig) mutation (Kermicle 1969, Science 166:1422-1424). The disclosures of which are incorporated herein by reference for this purpose. Methods for obtaining haploid plants are also disclosed in Kobayashi, M. et al., 1980, J. Heredity 71(1):9-14; Pollacsek, M., 1992, Agronomie (Paris) 12(3):247-251; Cho-Un-Haing et al., 1996, J. Plant Biol., 39(3):185-188; Genetic Manipulation in Plant Breeding, Proceedings International Symposium Organized by EUCARPIA, Sep. 8-13, 1985, Berlin, Germany; Chalyk et al., 1994, Maize Genet Coop. Newsletter 68:47. Souvre, A., et al. “Transformation of rape (Brassica Napes L.) through the haploid embryogenesis pathway”, ACTA SOCIETATIS BOTANICORUM POLONIAE, 1996, pp. 194-195, vol. 65, Nos. 1-2. Qing, Y. A., et al. “Biolistic transformation of haploid isolated microspores of barley”, Genome, 1997, pp. 570-581, vol. 40. No. 4. Abstract Only.

Haploid plants may also result from wide hybridization followed by chromosome elimination. Wide hybridization was used by crossing common barley, Hordeum vulgare with H. bulbosum and the subsequent elimination of H. bulbosum chromosomes. Wide hybridization has been used to develop barley, wheat, maize, sorghum, and millet cultivars. Another method for generating doubled haploid plants is gynogenesis. Gynogenesis involves the culture of female cells such as unfertilized ovaries or ovules (See, e.g., U.S. Pat. No. 5,492,827).

One critical aspect of the methods for inducing embryo formation from microspores is to disrupt and shift the microspore developmental process using physical or chemical means. The disruption and shift must coincide with the developmental stage of the microspore that subsequently allows embryo formation. Typically the stage that is disrupted is the late uninucleate to early bi-nucleate stage of development (Gaillard et al., 1991; Kott et al., 1988; Fan et al., 1988). Historically, the chief agent for disruption was elevated temperatures, (Keller et al., 1978; Cordewener et al., 1994) but chemicals such as colchicine, cytochalasin B, and trifluralin that are known to disturb cellular cytoskeleton organization have more recently been shown to be effective as well (See e.g., U.S. Pat. Nos. 5,900,375; 6,200,808). The nutrient medium is another aspect that has been shown to be important for recovery of embryos from induced microspores. Both the mineral composition of the medium and the percent of carbohydrates have been shown to be critical factors for some applications. High concentrations of sucrose (e.g., 13%) or other specific sugars such as maltose have been shown to be important. However, the optimal composition of the medium for embryo induction differs greatly from species to species. In addition to sugars and salts, plant growth regulators such as auxins, cytokinins and/or gibberellins may be required. Various gametocidal chemicals such as 2-hydroxynicotinic acid, 2-chloroethyl-phosphonic acid, and pronamide as well as undefined natural factors emanating from ovules (See e.g., U.S. Pat. Nos. 6,764,854; 6,362,393) may also be required components of the optimal nutrient medium. In U.S. Pat. No. 4,840,906, spikes containing anthers were pretreated at 4.degree. C. for a period of up to 28 days prior to culture of the barley microspores on media with varying sugar composition. This revealed the stimulative effect of maltose on the barley microspores. In U.S. Pat. Nos. 5,322,789 and 5,445,961, where isolated microspore and anther cultures of corn involved pre-treatment of microspores at 10.degree. C., the requirement for mannitol and the chromosome doubling agent colchicine in the culture medium was demonstrated. These and other methods developed for cereal crops have the limitation that the methods may result in formation of significant numbers of albino plants. U.S. Pat. No. 6,362,393 discloses a method for the production of doubled-haploid plants from wheat involved subjecting developing microspores to temperature and nutrient stress. A medium comprised of mannitol, maltose, auxins, cytokinins and/or gibberellin plant growth regulators, as well as a specific sporophytic development inducing chemical, were required for optimal embryo development. U.S. Pat. No. 6,764,854 describes an application of the above method for the production of doubled-haploid rice. U.S. Pat. No. 6,812,028 demonstrates a method for regeneration of isolated barley microspores that includes low temperature pretreatment, arabinogalactan protein, auxins and unknown natural factors from ovaries. Isolated microspore culture protocols have been described for various Brassica species, (Ferrie et al., 1995, 1999, 2004; Barro et al. 1999; and Lionneton 2001). Factors that have been identified that contribute to induction and development of microspore-derived embryos included growth conditions of the parent plants, stage of microspore development, temperature stress, osmotic stress, and carbohydrate composition of the medium. The requirement for temperature stress may be replaced by chemical inhibitors of cytoskeleton integrity (See, e.g., U.S. Pat. Nos. 5,900,375 and 6,200,808).

### ADDITIONAL REFERENCES

1. Arnison, P. G., et al. Genotype specific response of cultured broccoli (B. oleracea var italica) anthers to cytokinins. Plant Cell, Tissue and Organ Culture 20:217-222. (1990) 2. Barro, F., et al. Response of different genotypes of Brassica carinata to microspore culture, Plant Breeding 118: 79-81. (1999) 3. Brossa C. Biological effects of brassinosteroids. Critical Reviews in Biochemistry and Molecular Biology 34:339-358. (1999) 4. Chu, C. C. The N6 medium and its applications to anther culture of cereal crops. Science Press 43-50. (1978) 5. Cordwener, J. H. G., et al. Induction of microspore embryogenesis in Brassica napus L. is accompanied by specific changes in protein synthesis. Planta. 195:50-56. (1994). 6. Dunwell, J. M. Pollen, ovule and embryo culture as tools in plant breeding. In: Plant Tissue Culture and its Agricultural Applications, L. A. Withers and P. G. Alderson (eds) Butterworth, London. (1986) 7. Fabijanski, S. F., et al. Heat shock response during anther culture of broccoli (B. oleracea var italica). Plant Cell. Tissue and Organ Culture 26:203-212. (1991) 8. Fan, Z., et al. Development of microspores in vivo and in vitro in Brassica napus L. Protoplasma. 147:191-199. (1988). 9. Ferric, A. M. R., et al. Effects of brassinosteroids on microspore embryogenesis in Brassica species. In Vitro Cellular and Developmental Biology Plant 41: 742-745 (2005). 10. Ferric, A. M. R., et al. Biotechnology applications for haploids. In: Biotechnological Applications of Plant Cultures. Shargool, P. D. and Ngo, T. T., (eds.), CRC Press, Boca Raton, p 77-110. (1994) 11. Ferrie, A. M. R., et al. Evaluation of Brassica rapa L. genotypes for microspore culture response and identification of a highly embryogenic line. Plant Cell Reports 14: 580-584. (1995) 12. Fete, A. M. R., et al. Haploid Embryogenesis. In: In Vitro Embryogenesis in Plants, pp 309-344. (1995) 13. Ferrie, A. M. R. et al. Microspore culture for haploid plant production. In: O. L. Gamborg, and G. G. Phillips (eds.) Plant Cell, Tissue and Organ Culture: fundamental methods. Springer. Berlin, pp. 155-164. (1995) 14. Ferrie, A. M. R., et al. Brassica improvement through microspore culture. Biotechnology in Agriculture and Forestry Vol. 54: Brassica. E. C. Pua and C. J. Douglas. Berlin, Springer-Verlag: 149-168. (2004) 15. Ferrie, A. M. R., et al. Microspore embryogenesis of high sn-2 erucic acid Brassica oleracea germplasm. Plant Cell, Tissue and Organ Culture 57: 79-84. (1999) 16. Gaillard, A., et al. Optimization of maize microspore isolation and culture conditions for reliable plant regeneration. Plant Cell Reports 10:55-58. (1991) 17. Gamborg, O. L., et al. Nutrient requirements of suspension cultures of soybean root cells. Exp. Cell Res. 50:151-158. (1968) 18. Grove. M. D., et al. Brassinolide, a plant growth promoting steroid isolated from Brassica napus pollen. Nature 281:216-217. (1979) 19. Kao, K. N., et al. Nutritional requirements for growth of Vicia hajastana cells and protoplasts at a very low population density in liquid media. Planta 126:105-110. (1975) 20. Keller W. A., et al. High frequency production of microspore-derived plants from Brassica napus anther cultures. Z. Pflanzenzuchtg. 80:100-108. (1978) 21. Kott, L. S., et al. Cytological aspects of isolated microspore culture of Brassica napus. Can. J. Bot. 66:1658-1664. (1988) 22. Krishna, P. Brassinosteroid-mediated stress responses. J. Plant Growth Reg. 22:289-297. (2003) 23. Lichter, R. Induction of haploid plants from isolated pollen of Brassica napus. Z. Pflanzenphysiol 105:427-434. (1982) 24. Lionneton, E., et al. Improved microspore culture and doubled-haploid plant regeneration in the brown condiment mustard (Brassica juncea). Plant Cell Reports 20:126-130. (2001) 25. Lu Z., et al. Effect of brassinolide on callus growth and regeneration in Spartina patens (Poaceae). Plant Cell, tissue and Organ Culture 73:87-89 (2003) 26. Maheshwari, S. C., et al. Haploids from pollen grains—retrospect and prospect. Amer. J. Bot. 69: 865-879. (1982) 27. Nakajima, N., et al. Effects of brassinosteroid on cell division and colony formation of Chinese cabbage mesophyll protoplasts. Japanese Journal of Crop Science 65:114-118. (1996) 28. Oh. M. H., et al. Brassinolide affects the rate of cell division in isolated leaf protoplasts of Petunia hybrida. Plant Cell Reports 17:921-924. (1998) 29. Polsoni, L., et al. Large-scale microspore culture technique for mutation-selection studies in Brassica napus, Canadian Journal of Botany 66:1681-1685. (1988) 30. Pullman, G. S., et al. Brassinolide improves embryogenic tissue initiation in conifers and rice. Plant Cell Reports 22:96-104 (2003) 31. Nitsch, J. P., et al., Haploid plants from pollen grains. Science 163:85-87. (1969) 32. Saskaki, R. Brassinolide promotes adventitious shoot regeneration from cauliflower hypocotyl segments. Plant Cell, tissue and Organ Culture 71:111-116. (2002) 33. Sasse, J. M. Physiological actions of brassinosteroids. Brassinosteroids: Steroidal Plant Hormones. A. Sakurai, T. Yokota and S. D. Clouse. Tokyo, Springer-Verlag: 137-161. (1999) 34. Wilen, R., et al. Effects of 24-epibrassinolide on freezing and thermotolerance of bromegrass (Bromus inermis) cell cultures. Physiologia Plantarum 95:195-202. (1995) 35. Zhao J P and Simmonds D H and Newcomb W. High frequency production of doubled-haploid plants of Brassica napus cv. Topas derived from colchicine-induced microspore embryogenesis without heat shock. Plant Cell Rep. 15: 668-671 (1996).

Alemanno and Guiderdoni. “Increased doubled haploid plant regeneration from rice (Oryza sativa L.) anthers cultured on colchicine-supplemented media,” Plant Cell Rep., 13:432-436, 1994. Buter, “In vitro haploid production in maize,” In: In vitro haploid production in higher plants, Jain, et al., Veilleux (eds.), Kluwer Academic Publishers, Dordrecht, The Netherlands, 37-71, 1997. Chalyk, “Obtaining fertile pollen in maize maternal haploids,” Maize Genet. Coop. Newslett. 74, 17-18, (www.agron.missouri.edu/mnL/74/93chalyk.html), 2000. Coe, “A line of maize with high haploid frequency,” Am Nat, 93(873):381-382, 1959. Eder and Chalyk, “In vivo haploid induction in maize,” Theor. Appl. Genet., 104:703-708, 2002. Gayen et al., “Chromosome doubling in haploids through colchicine,” Maize Genet. Coop. Newslett, 68:65, (www.agron.missouri.edu/mn/68/101gayen.html), 1994. Kasha et al, “Haploids in Cereal Improvement: Anther and Microscope Culture”, Gene Manipulation in Plant Improvement II, Plenum Press, pp. 213-230. Armstrong, T. A., S. G. Metz and P. N. Mascia, “Two Regeneration Systems for the Production of Haploid Plants from Wheat Anther Culture,” Plant Science, vol. 51, pp. 231-237 (1987). Devaux, P., “Comparison of Anther Culture and the Hordeum bulbosum Method for the Production of Doubled Haploids in Winter Barley,” Plant Breeding, vol. 100, pp. 181-187 (1988). Heberle-Bors. E., “In vitro haploid formation from pollen: a critical review,” Theoretical and Applied Genetics, vol. 71, pp. 361-374 (1985). Picard, E., C. Hours, S. Gregoire, T. H. Phan and J. P. Meunier, “Significant improvement of androgenetic haploid and doubled haploid induction from wheat plants treated with a chemical hybridization agent,” Theoretical and Applied Genetics, vol. 74, pp. 289-297 (1987). Zhou, Huaping, and C. F. Konzak, “Improvement of Anther Culture Methods for Haploid Production in Wheat,” Crop Sci., vol. 29, pp. 817-821 (1989). U.S. Pat. No. 6,812,028, incorporated herein by reference in its entirety. U.S. Pat. No. 6,764,854, incorporated herein by reference in its entirety. U.S. Pat. No. 6,200,808, incorporated herein by reference in its entirety. U.S. Pat. No. 6,362,393, incorporated herein by reference in its entirety. U.S. Pat. No. 5,322,789, incorporated herein by reference in its entirety. U.S. Pat. No. 4,835,339, incorporated herein by reference in its entirety. U.S. Pat. No. 7,135,615, incorporated herein by reference in its entirety. Moeller C., M. Iqbal and G. Roebellen (1994) Efficient production of doubled haploid Brassica napus plants by colchicine treatment of microspores, Euphytica 75 (1-2) 95-104. Poelman, J. M. ‘Breeding Field Crops’ 1986 pp. 102-106: “Haploidy”; pp. 157-167: Anther culture and haploid plant production. Genovesi, A. D. In Biotechnology in Agriculture and Forestry 12. Haploids in Crop Improvement I. Bajaj, Y. P. S., ed. Springer-Verlag, N.Y., N.Y. Ch. 11.3:176-203. October 1990. M. Pollacsek, Plant improvement, Management of the ig gene for haploid induction in maize, (Received Apr. 1, 1991; accepted Jan. 16, 1992), Agronomie (1991) 12, 247-25 1. Y. Wan, et al., Efficient production of doubled haploid plants through colchicine treatment of anther-derived maize callus, Accepted Nov. 28, 1988, Theor Appl Genet (1989) 77: 889-892. Cho, Un-Haing, et al., Detection of Genetic Variation and Gene Introgression in Potato Dihaploids Using Randonly Amplified Polymorphic DNA (RAPD) Markers, J. Plant Biol. 39(3): 185-188 (1996). Mitsue Kobayashi, et al. Haploid induction and its genetic mechansim in alloplasmic common wheat. The Journal of Heredity 71: 9-14, 1980. Zhang et. al, J Genet Genomics. 2011 February; 38(2):89-94. doi: 10.1016/j.jcg.2011.01.004. Epub 2011 Feb. 23. Islam and Tuteja. Plant Sci. 2012 January; 182:134-44. doi: 10.1016/j. plantsci.2011.10.001. Epub 2011 Oct. 10. Germana. Plant Cell Rep. 2011 May; 30(5):839-57. doi: 10.1007/s00299-011-1061-7. Epub 2011 Mar. 24. Segui-Simarro et. al., Plant Cell Rep. 2011 May; 30(5):765-78. doi: 10.1007/s00299-010-0984-8. Epub 2010 Dec. 30. Noyes. Bioessays. 2008 September; 30(9):798-801. doi: 10.1002/bies.20795.

### Plant Transformation Methods.

Any of the recombinant DNA constructs provided herein can be introduced into the chromosomes of a host plant via methods such as Agrobacterium-mediated transformation, Rhizobium-mediated transformation, Sinorhizobium-mediated transformation, particle-mediated transformation, DNA transfection, DNA electroporation, or “whiskers”-mediated transformation. Aforementioned methods of introducing transgenes are well known to those skilled in the art and are described in U.S. Patent Application No. 20050289673 (Agrobacterium-mediated transformation of corn), U.S. Pat. No. 7,002,058 (Agrobacterium-mediated transformation of soybean), U.S. Pat. No. 6,365,807 (particle mediated transformation of rice), and U.S. Pat. No. 5,004,863 (Agrobacterium-mediated transformation of cotton), each of which are incorporated herein by reference in their entirety. Methods of using bacteria such as Rhizobium or Sinorhizobium to transform plants are described in Broothaerts, et al., Nature. 2005, 10; 433(7026):629-33. It is further understood that the recombinant DNA constructs can comprise cis-acting site-specific recombination sites recognized by site-specific recombinases, including Cre, Flp, Gin, Pin, Sre, pinD. Int-B13, and R. Methods of integrating DNA molecules at specific locations in the genomes of transgenic plants through use of site-specific recombinases can then be used (U.S. Pat. No. 7,102,055). Those skilled in the art will further appreciate that any of these gene transfer techniques can be used to introduce the recombinant DNA constructs into the chromosome of a plant cell, a plant tissue or a plant.

Many reports have been issued for production of transformed plants: Patent Publication No. 1: WO98/54961 Patent Publication No. 2: WO02/12520 Patent Publication No. 3: WO02/12521 Patent Publication No. 4: WO2005/017169 Patent Publication No. 5: WO2005/017152 Patent Publication No. 6: WO2007/069643 Non-patent Publication No. 1: De Cleene, M. and De Ley, J. (1976) The host range of crown gall. Bot. Rev. 42:389-466. Non-patent Publication No. 2: Grimsley, N., Horn, T., Davis, J. W. and Horn, B. (1987) Agrobacterium-mediated delivery of infectious maize streak virus into maize plants. Nature 325:177-179. Non-patent Publication No. 3: Grimsley, N. H., Ramos, C., Hein, T. and Horn, B. (1988) Meristematic tissues of maize plants are most susceptible to Agroinfection with maize streak virus. Bio/tecnology 6:185-189. Non-patent Publication No. 4: Grimsley, N., Horn, B., Ramos, C., Kado, C. and Rogowsky, P. (1989) DNA transfer from Agrobacterium to Zea mays or Brassica by agroinfection is dependent on bacterial virulence functions. Mol. Gen. Genet. 217:309-316. Non-patent Publication No. 5: Gould, J., Devey, M., Hasegawa, O., Ulian, E. C., Peterson, G. and Smith, R. H. (1991) Transformation of Zea mays L. using Agrobacterium tumefaciens and shoot apex. Plant Physiol. 95:426-434. Non-patent Publication No. 6: Mooney, P. A., Goodwin, P. B., Dennis, E. S, and Llewellyn, D. J. (1991) Agrobacterium tumefaciens-gene transfer into wheat tissues. Plant Cell, Tissues and Organ Culture 25:209-218. Non-patent Publication No. 7: Raineri, D. M., Bottino, P., Gordon, M. P. and Nester, E. W. (1990) Agrobacterium-mediated transformation of rice (Oryza sativa L.). Bio/technology 8:33-38. Non-patent Publication No. 8: Potrycus, I (1990) Gene transfer to cereals: an assessment. Bio/technology 8:535-542. Non-patent Publication No. 9: Chan, M-T., Chang, H-H., Ho, S-L., Tong, W-F. and Yu, S-M. (1993) Agrobacterium-mediated production of transgenic rice plants expressing a chimeric .alpha.-amylase promoter/.beta.-glucuronidase gene. Plant Mol. Biol. 22:491-506. Non-patent Publication No. 10: Hiei, Y., Ohta, S., Komari, T. and Kumashiro, T. (1994) Efficient transformation of rice (Oryza sativa L.) mediated by Agrobacterium and sequence analysis of the boundaries of the T-DNA. The Plant Journal 6:271-282. Non-patent Publication No. 11: Ishida, Y., Saito, H., Ohta, S., Hici, Y., Komari, T. and Kumashiro, T. (1996) High efficiency transformation of maize (Zea mays L.) mediated by Agrobacterium tumefaciens. Nature Biotechnology 14:745-750. Non-patent Publication No. 12: Cheng, M., Fry, J. E., Pang, S., Zhou, H., Hironaka, C. M., Duncan, D. R., Conner, T. W., Wan, Y. (1997) Genetic transformation of wheat mediated by Agrobacterium tumefaciens. Plant Physiol. 115: 971-980. Non-patent Publication No. 13: Tingay, S., McElroy, D., Kalla, R., Fieg, S., Wang, M., Thornton, S., Brettell, R. (1997) Agrobacterium tumefaciens-mediated barley transformation. Plant J. 11: 1369-1376. Non-patent Publication No. 14: Zhao, Z.-Y., Cai, T., Tagliani, L., Miller, M., Wang, N., Peng. H., Rudert, M., Schoeder. S., Hondred, D., Seltzer, J., Pierce, D. (2000) Agrobacterium-mediated sorghum transformation. Plant Mol. Biol. 44: 789-798. Non-patent Publication No. 15: Deji, A., Sakakibara, H., Ishida, Y., Yamada, S., Komari, T., Kubo, T., Sugiyama, T. (2000) Genomic organization and transcriptional regulation of maize ZmRR1 and ZmRR2 encoding cytokinin-inducible response regulators. Biochim. et Biophys. Acta 1492: 216-220. Non-patent Publication No. 16: Negrotto, D., Jolley, M., Beer, S., Wenck, A. R., Hansen, G. (2000) The use of phosphomannose-isomerase as a selection marker to recover transgenic maize plants (Zea mays L.) via Agrobacterium transformation. Plant Cell Reports 19: 798-803. Non-patent Publication No. 17: Nomura, M., Sentoku, N., Nishimura, A., Lin, J-H., Honda, C., Taniguchi, M., Ishida, Y., Ohta, S., Komari, T., Miyao-Tokumori, M., Kono-Murakami, Y., Tajima, S., Ku, M. S. B., Matsuoka, M. (2000a) The evolution of C4 plants: acquisition of cis-regulatory sequences in the promoter of C4-type pyruvate, orthophosphate dikinase gene. Plant J. 22: 211-221. Non-patent Publication No. 18: Nomura, M., Katayama, K., Nishimura, A., Ishida, Y., Ohta, S., Komari, T., Miyao-Tokutomi, M., Tajima, S., Matsuoka, M. (2000b) The promoter ofrbcS in a C3 plant (rice) directs organ-specific, light-dependent expression in a C4 plant (maize), but does not confer bundle sheath cell-specific expression. Plant Mol. Biol. 44: 99-106. Non-patent Publication No. 19: Taniguchi, M., Izawa, K., Ku, M. S. B., Lin, J-H., Saito, H., Ishida, Y., Ohta, S., Komari, T., Matsuoka, M., Sugiyama, T. (2000) The promoter for the maize C.sub.4 pyruvate, orthophosphate dikinase gene directs cell- and tissue-specific transcription in transgenic maize plants. Plant Cell Physiol. 41: 42-48. Non-patent Publication No. 20: Zhao, Z.-Y., Gu, W., Cai, T., Tagliani, L., Hondred, D., Bond, D., Schroeder, S., Rudert, M., Pierce, D. (2001) High throughput genetic transformation mediated by Agrobacterium tumefaciens in maize. Mol. Breed. 8: 323-333. Non-patent Publication No. 21: Frame, B. R., Shou, H., Chikwamba, R. K., Zhang, Z., Xiang, C., Fonger, T. M., Pegg, S. E. K., Li, B., Nettleton, D. S., Pei, D., Wang, K. (2002) Agrobacterium tumefaciens-mediated transformation of maize embryos using a standard binary vector system. Plant Physiol. 129: 13-22. Non-patent Publication No. 22: Ishida, Y., Saito, H., Hiei, Y., Komari, T. (2003) Improved protocol for transformation of maize (Zea mays L.) mediated by Agrobacterium tumefaciens. Plant Biotechnology 20:57-66. Non-patent Publication No. 23: Zhang, W., Subbarao, S., Addae, P., Shen, A., Armstrong, C., Peschke, V., Gilbertson, L. (2003) Cre/lox-mediated marker gene excision in transgenic maize (Zea mays L.) plants. Theor. Appl. Genet. 107: 1157-1168. Non-patent Publication No. 24: Frame, B. R., McMurray, J. M., Fonger. T. M., Main, M. L., Taylor, K. W., Torney. F. J., Paz, M. M., Wang, K. (2006) Improved Agrobacterium-mediated transformation of three maize inbred lines using MS salts. Plant Cell Rep. 25: 1024-1034. Non-patent Publication No. 25: Hiei, Y., Ishida, Y., Kasaoka, K. & Komari, T. Improved frequency of transformation in rice and maize by treatment of immature embryos with centrifugation and heat prior to infection with Agrobacterium tumefaciens. Plant Cell, Tissue and Organ Culture 87, 233-243 (2006). Non-patent Publication No. 26: Hiei, Y. & Komari, T. Improved protocols for transformation of indica rice mediated by Agrobacterium tumefaciens. Plant Cell, Tissue and Organ Culture 85, 271-283 (2006). Non-patent Publication No. 27: Komari, T. Transformation of callus cultures of nine plant species mediated by Agrobacterium. Plant Sci. 60, 223-229 (1989). Non-patent Publication No. 28: Bent, A. F. (2000) Arabidopsis in planta transformation. Uses, mechanisms, and prospects for transformation of other species. Plant Physiol., 124:1540-1547. Non-patent Publication No. 29: Bevan, M. W., Flavell, R. B., Chilton, M.-D. (1983) A chimaeric antibiotic resistance gene as a selectable marker for plants cell transformation. Nature, 304:184-187. Non-patent Publication No. 30: Comai, L., Facciotti, D., Hiatt, W. R., Thompson, G., Rose, R. E., Stalker, D. M. (1985) Expression in plants of a mutant aroA gene from Salmonella typhimurium confers tolerance to glyphosate. Nature, 317:741-744. Non-patent Publication No. 31: De Block, M., Botterman, J., Vandewiele, M., Dockx, J., Thoen, C., Gossele, V., Movva, N. R., Thompson, C., Van Montagu, M., Leemans, J. (1987) Engineering herbicide resistance in plants by expression of a detoxifying enzyme. EMBO J., 6:2513-2518. Non-patent Publication No. 32: Ebinuma, H., Sugita, K., Matsunaga, E., Yamakado, M. (1997) Selection of marker-free transgenic plants using the isopentenyl transferase gene as a selectable marker. Proc. Natl. Acad. Sci. U.S.A., 94:2117-2121. Non-patent Publication No. 33: Elliott, A. R., Campbell, J. A., Brettell R. I. S., Grof, C. P. L. (1998) Agrobacterium-mediated transformation of sugarcane using GFP as a screenable marker. Aust. J. Plant Physiol., 25:739-743. Non-patent Publication No. 34: Erikson, O. S., Hertzberg, M., Nasholm, T. (2005) The dsdA gene from Escherichia coli provides a novel selectable marker for plant transformation. Plant Mol. Biol., 57:425-433. Non-patent Publication No. 35: Gleave, A. P., Mitra, D. S., Mudge, S. R., Morris, B. A. M. (1999) Selectable marker-free transgenic plants without sexual crossing: transient expression of cre recombinase and use of a conditional lethal dominant gene. Plant. Mol. Biol., 40:223-235. Non-patent Publication No. 36: Haldrup, A., Petersen, S. G., Okkels, F. T. (1998) The xylose isomerase gene from Thermoanaerobacterium thermosulfurogenes allows effective selection of transgenic plant cells using D-xylose as the selection agent. Plant Mol. Biol., 37:287-296. Non-patent Publication No. 37: Joersbo, M., Donaldson, I., Kreiberg, J., Petersen, S. G., Brunstedt, J., Okkels, F. T. (1998) Analysis of mannose selection used for transformation of sugar beet. Mol. Breed., 4:111-117. Non-patent Publication No. 38: Komari, T., Hiei, Y., Saito, Y., Murai, N., Kumashiro, T. (1996) Vectors carrying two separate T-DNAs for co-transformation of higher plants mediated by Agrobacterium tumefaciens and segregation of transformants free from selection markers. The Plant Journal, 10:165-174. Non-patent Publication No. 39: Kuiper, H. A., Meter, G. A., Noteborn, H. P. J. M., Kok, E. J. (2001) Assessment of the food safety issues related to genetically modified food. The Plant Journal, 27:503-528. Non-patent Publication No. 40: Potrykus, I., Bilang, R., Futterer J., Sautter, C., Schrott, M. (1998) Genetic engineering of crop plants. Agricultural Biotechnology, Marcel Deker Inc., New York. 119-159. Non-patent Publication No. 41: Tetsuo Takematsu (1982), Josozai Kenkyu Soran (Overview of Herbicide Research), Hakuyu-sha, Tokyo, 79-154 (in Japanese). Non-patent Publication No. 42: Waldron, C., Murphy, E. B., Roberts, J. L., Gustafson, G. D., Armour, S. L., Malcolm, S. K. (1985) Resistance to hygromycin B: A new marker for plant transformation studies. Plant Mol. Biol., 5:102-108. Non-patent Publication No. 43: Zhu, Y. J., Asbayani, R., Moore, P. H. (2004) Green fluorescent protein as a visual selection marker for papaya (Carica papaya L.) transformation. Plant Cell Rep., 22:660-667. Non-patent Publication No. 44: Chu, C.-C. (1978) The N6 medium and its applications to anther culture of cereal crops. In Proc. Symp. Plant Tissue Culture. Peking: Science Press, pp. 43-50. Non-patent Publication No. 45: Sambrook, J., Fritsch, E. F., Maniatis, T. (1989) Molecular Cloning: A Laboratory Manual, 2nd ed., Cold Spring Harbor Laboratory Press, Cold Spring Harbor, N.Y.

### Methods for Trans-Silencing Genes.

In certain embodiments, suppression of a MSH1 gene in a plant is effected with a transgene. Transgenes that can be used to suppress MSH1 expression include, but are not limited to, transgenes that produce dominant-negative mutants, a small inhibitory RNA (siRNA), a microRNA (miRNA), a hairpin RNA, a co-suppressing sense RNA, and/or an anti-sense RNA that provide for inhibition of the MSH1 target gene. U.S. patents incorporated herein by reference in their entireties that describe suppression of endogenous plant genes by transgenes include U.S. Pat. No. 7,109,393, U.S. Pat. No. 5,231,020 and U.S. Pat. No. 5,283,184 (co-suppression methods); and U.S. Pat. No. 5,107,065 and U.S. Pat. No. 5,759,829 (antisense methods). In certain embodiments, transgenes specifically designed to produce double-stranded RNA (dsRNA) molecules or Virus Induced Gene Silencing (VIGS) with homology to MSH1 can be used to decrease expression as described in U.S. Provisional 61/901,349, which is incorporated here in its entirety.

### Methods for Mutating Genes in Plants.

Mutations in chromosomal genes within the plant cell can be made and identified by a variety of methods. In certain embodiments where MSH1 suppression is achieved by use of a mutation in the endogenous MSH1 gene of a plant, the presence or absence of that mutation in the genomic DNA can be readily determined by a variety of techniques. Certain techniques can also be used that provide for identification of the mutation in a hemizygous state (i.e. where one chromosome carries the mutated msh1 gene and the other chromosome carries the wild type MSH1 gene). Mutations in MSH1 DNA sequences that include insertions, deletions, nucleotide substitutions, and combinations thereof can be detected by a variety of effective methods including, but not limited to, those disclosed in U.S. Pat. Nos. 5,468,613, 5,217,863; 5,210,015; 5,876,930; 6,030,787; 6,004,744; 6,013,431; 5,595,890; 5,762,876; 5,945,283; 5,468,613; 6,090,558; 5,800,944; 5,616,464; 7,312,039; 7,238,476; 7,297,485; 7,282,355; 7,270,981 and 7,250,252 all of which are incorporated herein by reference in their entireties. For example, mutations can be detected by hybridization to allele-specific oligonucleotide (ASO) probes as disclosed in U.S. Pat. Nos. 5,468,613 and 5,217,863. U.S. Pat. No. 5,210,015 discloses detection of annealed oligonucleotides where a 5′ labelled nucleotide that is not annealed is released by the 5′-3′ exonuclease activity. U.S. Pat. No. 6,004,744 discloses detection of the presence or absence of mutations in DNA through a DNA primer extension reaction. U.S. Pat. No. 5,468,613 discloses allele specific oligonucleotide hybridizations where single or multiple nucleotide variations in nucleic acid sequence can be detected by a process in which the sequence containing the nucleotide variation is amplified, affixed to a support and exposed to a labeled sequence-specific oligonucleotide probe. Mutations can also be detected by probe ligation methods as disclosed in U.S. Pat. No. 5,800,944 where sequence of interest is amplified and hybridized to probes followed by ligation to detect a labeled part of the probe. U.S. Pat. Nos. 6,613,509 and 6,503,710, and references found therein provide methods for identifying mutations with mass spectroscopy. These various methods of identifying mutations are intended to be exemplary rather than limiting as the methods of the present invention can be used in conjunction with any polymorphism typing method to identify the presence of absence of mutations in MSH1 target gene in genomic DNA samples. Furthermore, genomic DNA samples used can include, but are not limited to, genomic DNA isolated directly from a plant, cloned genomic DNA, or amplified genomic DNA. The use of mutations in endogenous MSH1 genes is specifically provided herein.

Mutations in endogenous plant MSH1 genes can be obtained from a variety of sources and by a variety of techniques. A homologous replacement sequence containing one or more loss of function mutations in the MSH1 gene and homologous sequences at both ends of the double stranded break can provide for homologous recombination and substitution of the resident wild-type MSH1 gene sequence in the chromosome with a msh1 replacement sequence causing attenutated or loss of MSH1 function mutation(s). Such loss of function mutations include, but are not limited to, insertions, deletions, and substitutions of sequences within a MSH1 gene that result in either a complete loss of MSH1 gene function or a loss of MSH1 gene function sufficient to elicit alterations (i.e. heritable and reversible epigenetic changes) in other chromosomal loci or mutations in other chromosomal loci. Loss-of-function mutations in an MSH1 target gene include, but are not limited to, frameshift mutations, pre-mature translational stop codon insertions, deletions of one or more functional domains that include, but are not limited to, a DNA binding (Domain I), an ATPase (Domain V) domain, and/or a carboxy-terminal GIY-YIG type endonuclease domain, and the like. Methods for substituting endogenous chromosomal sequences by homologous double stranded break repair have been reported in tobacco and maize (Wright et al., Plant J. 44, 693, 2005; D'Halluin, et al., Plant Biotech. J. 6:93, 2008). A homologous replacement msh1 sequence (i.e. which provides a loss of function mutation in the MSH1 gene sequence) can also be introduced into a targeted nuclease cleavage site by non-homologous end joining or a combination of non-homologous end joining and homologous recombination (reviewed in Puchta, J. Exp. Bot. 56, 1, 2005; Wright et al., Plant J. 44, 693, 2005). In certain embodiments, at least one site specific double stranded break can be introduced into the MSH1 gene by a meganuclease. Genetic modification of meganucleases can provide for meganucleases that cut within a recognition sequence that exactly matches or is closely related to specific endogenous MSH1 gene sequence (WO/06097853A1, WO/06097784A1, WO/04067736A2, U.S. 20070117128A1). It is thus anticipated that one can select or design a nuclease that will cut within a MSH1 gene sequence. In other embodiments, at least one site specific double stranded break can be introduced in the MSH1 gene target sequence with a zinc finger nuclease. The use of engineered zinc finger nuclease to provide homologous recombination in plants has also been disclosed (WO 03/080809, WO 05/014791, WO 07014275, WO 08/021207). In other embodiments, a CRISPR/CAS9 RNA-guided gene targeting endonuclease system can be used (e.g., Wanner B L, Teramoto J, Mori H. Phys Life Rev. 2013 Dec. 12. pii: S1571-0645(13)00195-4. doi: 10.1016/j.plrev.2013.12.003; Puchta H, Fauser F. Gene targeting in plants: 25 years later. Int J Dev Biol. 2013; 57(6-8):629-37. doi: 10.1387/ijdb. 130194 hp). In still other embodiments, mutations in endogenous MSH1 genes can be identified through use of the TILLING technology (Targeting Induced Local Lesions in Genomes) as described by Henikoff et al. where traditional chemical mutagenesis would be followed by high-throughput screening to identify plants comprising point mutations or other mutations in the MSH1 target gene (Henikoff et al., Plant Physiol. 2004, 135:630-636). Alternatively, high throughput DNA sequencing on an Illumina HiSeq 2500 or equivalent machine on 3-D pools of indexed PCR products of MSH1 regions of mutagenized plants can be used to identify mutants in MSH1 genes in plants. The recovery of mutations in endogenous MSH1 genes is specifically provided herein.

### SUMMARY

A crop plant subjected to a method of suppressing MSH1 produces useful traits in plants or plants useful for plant breeding or plants comprising altered chromosomal loci as described in U.S. patent application Ser. No. 13/462,216, in U.S. patent application Ser. No. 14/454,518, in U.S. patent application Ser. No. 14/495,498, in U.S. patent application Ser. No. 14/536,135, in U.S. Provisional 61/863,267, in U.S. Provisional 61/882,140, in U.S. Provisional 61/901,349. U.S. Provisional 61/930,602, U.S. Provisional 61/970,424, and U.S. Provisional 61/980,096, each of which is incorporated herein by reference in its entirety. The present invention provides methods to create improved stability to these epigenetic modifications across many subsequent plant generations. The present invention uses the production of haploid or double haploids to accomplish this. These haploid or double haploid plants or plant cells are derived from a parental plant or cell that is heterozygous for epigenetic modifications, including, but not limited to, DNA methylation modifications. The starting parent's genome can be genetically homozygous or heterozygous, i.e., the diploid or polyploid genomic sequences can be homozygous or heterozygous for chromosomal DNA sequences. This method converts epigenetically heterozygous plants into plants that are epigenetically homozygous and thereby stabilizes these epigenetic modifications in subsequent generations.

In certain embodiments, a recessive mutation in a plant's MSH1 gene occurs in a genetically homozygous state to initiate epigenetic modifications (see U.S. patent application Ser. No. 13/462,216, which is incorporated herein by reference in its entirety). In order to restore both copies of the MSH1 gene to their normal non-mutant state, a cross to an isogenic line with a normal MSH1 gene is required, followed by selfing to identify progeny with useful traits and containing one or more normal MSH1 genes, preferably at least two normal MSH1 genes. Alternatively, a transgene encoding a functional MSH1 could be used to restore MSH1 function. Haploid or double haploid methods can be applied at any stage after F1 fertilization, from the F1 embryo or plant or its subsequent selfed generations (F2-Fn), preferably the F2, F3, F4, F5, or F6 generations to provide epigenetically homozygous lines that exhibit exceptional epigenetic stability and contain normal functional MSH1 genes.

In certain embodiments, a recessive mutation in a plant's MSH1 gene occurs in a genetically homozygous state to initiate epigenetic modifications (see U.S. patent application Ser. No. 13/462,216, which is incorporated herein by reference in its entirety). In order to restore both copies of the MSH1 gene to their normal non-mutant state, a cross to a genetically different line with a normal MSH1 gene is required, to produce a genetically and epigenetically heterogenous F1 progeny. Said progeny is selfed and screened to identify F2 progeny containing one or more normal MSH1 genes. Haploid or double haploid methods can be applied at any stage after F1 fertilization, from the F1 embryo or plant or its subsequent selfed generations (F2-Fn), preferably the F2, F3, F4, F5, or F6 generations to provide epigenetically homozygous lines that exhibit exceptional epigenetic stability and contain non-mutant MSH1 genes.

In certain embodiments, suppression of a plant's MSH1 gene initiates epigenetic modifications to produce useful traits (see U.S. patent application Ser. No. 13/462,216, U.S. Provisional 61/863,267, U.S. Provisional 61/882,140, and U.S. Provisional 61/901,349, each of which is incorporated herein by reference in its entirety). In order to restore MSH1 function, a plant heterozygous for the suppressing gene can be selfed or outcrossed to identify progeny that are not suppressed for MSH1 function. Haploid or double haploid methods can be applied at any stage after meiosis, for either pollen or egg cell derived gametophytic tissues or to the F1 fertilized embryo or plant or its subsequent selfed or outcrossed generations (F2-Fn or the equivalent outcross or backcross generation), preferably the F2, F3, F4, F5, or F6 generations, or equivalent generations if outcrossed or backcrossed, to provide epigenetically homozygous lines that exhibit improved epigenetic stability and lack the suppressing gene.

In certain embodiments a method for producing a haploid or double haploid plant or plant cell useful for plant breeding comprising the steps of: (a) crossing or self-pollinating or culturing gametophytic tissue from a plant, wherein said plant or a progenitor plant thereof is or had been subjected to MSH1 suppression; and, (b) producing a haploid or double haploid plant or plant cell from the crossing or self-pollination or culturing gametophytic tissue of step (a) or from progeny thereof, thereby producing a haploid or double haploid plant or plant cell useful for plant breeding is provided. In certain embodiments a method the haploid or double haploid plant is produced from progeny of the F1, F2, F3, F4, F5, F6 or S1, S2, S3, S4, S5 or S6 generations. In certain embodiments the method of producing a haploid or double haploid plant utilizes CenH3 suppression and/or a tailswap CenH3 protein. In certain embodiments the plant is a corn, rice, wheat, sorghum, barley, soybean, cotton, canola, sugarbeet, alfalfa, potato, or tomato plant. In certain embodiments the crossing of step (a) is between plants that are essentially isogenic.

In certain embodiments a method for identifying a double haploid plant harboring a useful trait comprising the steps of: (a) crossing a candidate plant to a second plant, wherein the candidate plant is a double haploid derived from: (i) a selfed plant wherein said selfed plant or a progenitor thereof is or had been subjected to MSH1 suppression; or (ii) a cross wherein at least one parental plant or a progenitor thereof is or had been subjected to MSH1 suppression; and, (b) identifying one or more progeny plants from the cross in step (a) that have a useful trait, to a greater extent than the candidate plant, the second plant, or a control plant, thereby identifying a double haploid plant harboring a useful trait is provided. In certain embodiments the control plant is progeny of a cross between: (i) a plant that is not progeny of a selfed plant, a crossed plant, or parent thereof that is or had been subjected to MSH1 suppression; and (ii) a plant that is isogenic to the second plant. In certain embodiments a plant part obtained from the plant or progeny thereof is provided. In certain embodiments the plant part is selected from the group consisting of a seed, leaf, stem, fruit, and a root. In certain embodiments a processed plant product obtained from the plant part. In certain embodiments a clonal propagate is obtained from the plant or plant part.

In certain embodiments a method for producing a haploid or double haploid plant or plant cell exhibiting new combinations of altered chromosomal loci useful for breeding comprising the steps of: (a) crossing or selfing or culturing gametophytic tissue from a plant comprising altered chromosomal loci induced by MSH1 suppression; (b) producing a haploid or double haploid plant or plant cell from the crossing or self-pollination or culturing gametophytic tissue of step (a) or from progeny thereof; and, (c) assaying said plant or plant cell of step (b) or progeny thereof to identify and select individuals with new combinations of altered chromosomal loci, thereby producing a haploid or double haploid exhibiting new combinations of altered chromosomal loci useful for breeding is provided. In certain embodiments the DNA methylation of one or more altered chromosomal loci occurs at CHG or CHH sites within one or more DNA regions selected from the group consisting of MSH1, pericentromeric regions, transposable elements, transposable elements containing genes, and transposable elements in pericentromeric regions. In certain embodiments one or more sRNAs assayed have sequence homology to one or more regions selected from the group consisting of MSH1, pericentromeric regions, transposable elements, transposable elements containing genes, and transposable elements in pericentromeric regions.

In certain embodiments a method for identifying a haploid or double haploid plant or plant cell with altered chromosomal loci useful for plant breeding comprising the steps of: (a) assaying one or more haploid or double haploid plants or plant cells subjected to MSH1 suppression or derived from a progenitor that was subjected to MSH1 suppression; and, (b) identifying one or more plants or plant cells from step (a) comprising one or more altered chromosomal loci, thereby identifying a haploid or double haploid plant or plant cell with altered chromosomal loci useful for plant breeding is provided. In certain embodiments the DNA methylation of one or more altered chromosomal loci occurs at CHG or CHH sites within one or more DNA regions selected from the group consisting of MSH1, pericentromeric regions, transposable elements, transposable elements containing genes, and transposable elements in pericentromeric regions. In certain embodiments one or more sRNAs assayed have sequence homology to one or more regions selected from the group consisting of MSH1, pericentromeric regions, transposable elements, transposable elements containing genes, and transposable elements in pericentromeric regions.

In certain embodiments a method for producing a haploid or double haploid plant or plant cell useful for plant breeding or for harboring a useful trait comprising the steps of: (a) culturing a plant cell, wherein said plant cell is or had been subjected to MSH1 suppression or is derived from a plant or a progenitor plant thereof that is or had been subjected to MSH1 suppression; and, (b) producing a haploid or double haploid plant from the plant cell of step (a) or from progeny thereof, thereby producing a haploid or double haploid plant or plant cell useful for plant breeding is provided herein.

In certain embodiments, a hybrid plant or plant seed, wherein at least one of the progenitor plants is a plant produced by any of the aforementioned methods, is provided.

In certain embodiments, a plant or plant seed, wherein at least one of the progenitor plants is a plant produced by any of the aforementioned methods, is provided.

Also provided are plants or progeny thereof that exhibit a useful trait that are made by any of the aforementioned methods. Plant parts obtained from the plant or progeny thereof made by any of the aforementioned methods are also provided. In certain embodiments, the part is selected from the group consisting of a seed, leaf, stem, fruit, and a root. Processed plant products obtained from the plant parts are also provided. Clonal propagates obtained from the plants, the progeny thereof, or from the plant parts are also provided.

In certain embodiments, the plant is selected from the group consisting of a crop plant, a tree, a bush, a grass, and a vine. In certain embodiments, the crop plant is selected from the group consisting of corn, soybean, cotton, canola, wheat, rice, tomato, tobacco, millet, potato, sugarbeet, cassava, alfalfa, barley, oats, sugarcane, sunflower, strawberry, and sorghum. In certain embodiments, the tree is selected from the group consisting of an apple, apricot, grapefruit, orange, peach, pear, plum, lemon, coconut, poplar, eucalyptus, date palm, palm oil, pine, and an olive tree. In certain embodiments, the bush is selected from the group consisting of a blueberry, raspberry, and blackberry bush. Also provided are plants or progeny thereof obtained by any of the aforementioned methods. Also provided are plant parts obtained from the plant or progeny thereof that were made by any of the aforementioned methods. In certain embodiments, the plant part is selected from the group consisting of a seed, leaf, stem, fruit, and a root. Also provided are clonal propagates obtained from the plant or progeny thereof that were made by any of the aforementioned methods.

### BRIEF DESCRIPTION OF THE DRAWINGS

None.

### DESCRIPTION

As used herein, the terms “useful for plant breeding” or “useful for breeding” refer to plants that are useful in a plant breeding program for the objective of developing improved plant traits such as improved yield, stress tolerance, improved mineral nutrition, and other traits of importance to plant breeders.

As used herein, the terms “pericentromeric” or “pericentromere” refer to heterochromatic regions containing abundant repeated sequences, transposable elements, and retrotransposons that physically flank the centromeric regions. At the sequence level, a functional definition for pericentromeric sequences are repeated sequences that contain transposable elements and retrotransposons embedded in said repeated sequences. When known, centromeric repeats can be computationally removed from the repeated sequences, but their presence is not detrimental if not computationally removed. When available, chromosomal positioning information about the location of sequences that are located adjacent to the centromere can be used as additional criteria for pericentromeric sequences.

As used herein, the terms “CG altered gene” or “CG altered genes” refer to a gene or genes with increased or decreased levels of DNA methylation (5meC) at CG nucleotides within or near a gene or genes. The region near a gene is within 5,000 bp, preferably within 1,000 bp, of either the 5′ or 3′ end of the gene or genes.

As used herein, the terms “CG enhanced genes” refers to CG altered genes with higher levels of DNA methylation or sRNA derived from said CG enhanced genes relative to the levels from a reference plant.

As used herein, the phrase “plant cell” refers to a plant cell capable of regenerating into a plant or a fertile plant.

As used herein, the phrases “haploid” or “double haploid” refer to a chromosome number that is one-half of the parental plant chromosome number when haploid, and a chromosome number the same as the parental plant when double haploid and wherein the double haploid is derived from a doubling the chromosomes from a haploid. Double haploid refers to the initial double haploid plant or plant cell and their subsequent generations of progeny.

The phrase “culturing gametophytic tissue” refers to the process of producing haploid and/or double haploid cells and/or tissues from male and/or female gametophytic tissues including, but not limited to androgenesis, anther culture, microspore culture, immature pollen, egg or ovule-ovary culture, pollination with irradiated pollen or pollen that does not transfer stably maintained male chromosomes, or other haploid plant production methods, such as the method of producing a haploid or double haploid plant utilizing CenH3 suppression and/or a tailswap CenH3 protein, known to those skilled in the art.

As used herein, the terms “CG depleted genes” refers to to CG altered genes with lower levels of DNA methylation or sRNA derived from said CG enhanced genes relative to the levels from a reference plant.

As used herein, the phrases “suppression” or “suppressing expression” of a gene refer to any genetic, nucleic acid, nucleic acid analog, environmental manipulation, grafting, transient or stably transformed methods of any of the aforementioned methods, or chemical treatment that provides for decreased levels of functional gene activity, including inhibition of the protein activity produced from the gene, in a plant or plant cell relative to the levels of functional gene activity that occur in an otherwise isogenic plant or plant cell that had not been subjected to this genetic or environmental manipulation.

As used herein, the phrases “subjected to MSH1 suppression” or “MSH1 suppression” refers to suppression of MSH1 expression, activity, or function, including RNAi suppression, gene mutations, and MSH1 mitochondrial-complementation, and including the methods referred to in the phrases “suppression” or “suppressing expression”.

As used herein, the phrase “MSH1 mitochondrial-complementation” refers to a method of targeting MSH1 to mitochondria in the presence of a lack of MSH1 in the plastids.

As used herein, the phrases “altered chromosomal loci” (used as singular or plural herein) or “altered chromosomal locus (singular) or “altered chromosomal loci induced by MSH1 suppression” refer to portions of a chromosome that have undergone a heritable epigenetic change relative to the corresponding parental chromosomal loci prior to MSH1 suppression. The altered chromosomal loci can occur in any of the generations of progeny derived from a progenitor plant subjected to MSH1 suppression. Heritable epigenetic changes in altered chromosomal loci include, but are not limited to, methylation of chromosomal DNA. and in particular, methylation of cytosine residues to 5-methylcytosine residues. As used herein, “chromosomal loci” refer to loci in chromosomes located in the nucleus of a cell. Altered chromosomal loci can be assayed for DNA methylation or sRNA derived from these regions.

Altered chromosomal loci have altered DNA methylation levels, and/or altered levels of sRNA derived from these regions, relative to the corresponding parental chromosomal loci prior to MSH1 suppression or to a parental chromosome in a lineage not subjected to MSH1 suppression.

As used herein, the phrase “new combinations of altered chromosomal loci” refers to nuclear chromosomal regions in a progeny plant with one or more differences in altered chromosomal loci when compared to altered chromosomal loci of a parental plant if derived by self-pollination, or if derived from a cross, when compared to either parental plant, each compared separately to said progeny plant.

As used herein, the phrases “assaying” or “assayed” refer to methods for determining the amounts, or sequences, or both, of DNA methylation or sRNA, corresponding to one or more nuclear chromosomal regions for DNA or with homology to one or more nuclear chromosomal regions for sRNA. The nuclear chromosomal regions assayed for DNA methylation can be a single nucleotide position or a region greater than this. Preferably the DNA methylation is from a region comprising one or more CG, CHG, or CHH sites and is compared to the corresponding parental chromosomal loci prior to MSH1 suppression. sRNA can be measured for a single type of sRNA, one or more sRNAs, or a whole population of sRNAs by methods known to those skilled in the art.

As used herein, the phrases “epigenetic” or “epigenetically modified” or epigenetic modifications” or “epigenetic modification” refer to genes, chromosomes, and genomes located in the nucleus that have undergone a heritable change in response to MSH1 suppression in a plant or progeny plant relative to the corresponding parental chromosomal loci prior to MSH1 suppression. Heritable changes in altered chromosomal loci include, but are not limited to, methylation of chromosomal DNA, and in particular, methylation of cytosine residues to 5-methylcytosine residues, and/or post-translational modification of histone proteins, and in particular, histone modifications that include, but are not limited to, acetylation, methylation, ubiquitinylation, phosphorylation, and sumoylation (covalent attachment of small ubiquitin-like modifier proteins). Changes in DNA methylation of a region are often associated with changes in sRNA levels with homology to the region.

As used herein, the phrase “progenitor” refers to an ancestral plant or plant cell from which the current plant or plant cell was derived.

As used herein, the term “reference plant” refers to a parental plant not subjected to MSH1 suppression or progenitor of a parental plant prior to MSH1 suppression, but otherwise isogenic to the candidate plant to which it is being compared. In a cross of two parental plants, a “reference plant” can also be from a parental plant wherein MSH1 suppression was not used in said parental plant or one of its progenitors.

As used herein, the phrases “increased DNA methylation” or “decreased DNA methylation” refer to nucleotides, regions, genes, chromosomes, and genomes located in the nucleus that have undergone a change in 5meC levels in a plant or progeny plant relative to the corresponding parental chromosomal loci prior to MSH1 suppression or to a parental plant not subjected to MSH1 suppression.

As used herein, the term “comprising” means “including but not limited to”.

As used herein, the phrase “crop plant” includes, but is not limited to, cereal, seed, grain, fruit, and vegetable crop plants.

As used herein, the term “commercially synthesized” or “commercially available” DNA refers to the availability of any sequence of up to 500 bp in length or longer from DNA synthesis companies that provide a DNA sample containing the sequence submitted to them. MSH1 regions for use in a hairpin DNA expression vector or CenH3 synthetic gene sequences or any desired DNA sequence can be obtained by commercial synthesis of the submitted sequence.

As used herein, the phrase “loss of function” refers to a diminished, partial, or complete loss of function.

As used herein, the phrase “mutated gene” or “gene mutation” refers to portions of a gene that have undergone a heritable genetic change in a nucleotide sequence relative to the nucleotide sequence in the corresponding parental gene that results in a reduction in function of the gene's encoded protein function. Mutations include, but are not limited to, nucleotide sequence inversions, insertions, deletions, substitutions, or combinations thereof. In certain embodiments, the mutated gene can comprise mutations that are reversible. In this context, reversible mutations in the chromosome can include, but are not limited to, insertions of transposable elements, defective transposable elements, and certain inversions. In certain embodiments, the gene comprises mutations are irreversible. In this context, irreversible mutations in the chromosome can include, but are not limited to, deletions.

As used herein, the term “heterotic group” refers to a group of genetically related germplasms that produce superior hybrids when crossed to genetically distinct germplasm of another heterotic group.

As used herein, the term “genetically homogeneous” or “genetically homozygous” refers to the two parental genomes provided to a progeny plant as being essentially identical at the DNA sequence level.

As used herein, the term “epigenetically homogeneous” or “epigenetically homozygous” refers to the two parental genomes or individual loci provided to a progeny plant as being essentially identical at the epigenetic level.

As used herein, the term “genetically heterogeneous” or “genetically heterozygous” refers to the two parental genomes provided to a progeny plant as being substantially different at the sequence level. That is, one or more genes from the male and female gametes occur in different allelic forms with DNA sequence differences between them.

As used herein, the term “epigenetically heterogeneous” or “epigenetically heterozygous” refers to the two parental genomes provided to a progeny plant as being substantially different at the epigenetic level. That is, one or more genes from the male and female gametes occur in different allelic forms with epigenetic differences between them.

As used herein, the term “isogenic” refers to the two plants that have essentially identical genomes at the DNA sequence levels level, independent of any DNA methylation or epigenetic differences. In certain instances “isogenic” refers to two plant lines essentially differing only in the presence or absence of DNA or RNA sequences causing suppression of MSH1 function.

As used herein, the term “F1” refers to the first progeny of two genetically or epigenetically different plants. “F2” refers to progeny from the self pollination of the F1 plant. “F3” refers to progeny from the self pollination of the F2 plant. “F4” refers to progeny from the self pollination of the F3 plant. “F5” refers to progeny from the self pollination of the F4 plant. “Fn” refers to progeny from the self pollination of the F(n−1) plant, where “n” is the number of generations starting from the initial F1 cross. Crossing to a isogenic line (backcrossing) or unrelated line (outcrossing) at any generation will also use the “Fn” notation, where “n” is the number of generations starting from the initial F1 cross.

As used herein, the term “S1” refers to a first selfed plant. “S2” refers to progeny from the self pollination of the S1 plant. “S3” refers to progeny from the self pollination of the S2 plant. “S4” refers to progeny from the self pollination of the S3 plant. “S5” refers to progeny from the self pollination of the S4 plant. “Sn” refers to progeny from the self pollination of the S(n−1) plant, where “n” is the number of generations starting from the initial S1 cross.

As used herein, the term “progeny” refers to any one of a first, second, third, or subsequent generation obtained from a parent plant.

The phrase “operably linked” as used herein refers to the joining of nucleic acid sequences such that one sequence can provide a required function to a linked sequence. In the context of a promoter, “operably linked” means that the promoter is connected to a sequence of interest such that the transcription of that sequence of interest is controlled and regulated by that promoter. When the sequence of interest encodes a protein and when expression of that protein is desired, “operably linked” means that the promoter is linked to the sequence in such a way that the resulting transcript will be efficiently translated. If the linkage of the promoter to the coding sequence is a transcriptional fusion and expression of the encoded protein is desired, the linkage is made so that the first translational initiation codon in the resulting transcript is the initiation codon of the coding sequence. Alternatively, if the linkage of the promoter to the coding sequence is a translational fusion and expression of the encoded protein is desired, the linkage is made so that the first translational initiation codon contained in the 5′ untranslated sequence associated with the promoter is linked such that the resulting translation product is in frame with the translational open reading frame that encodes the protein desired. Nucleic acid sequences that can be operably linked include, but are not limited to, sequences that provide gene expression functions (i.e., gene expression elements such as promoters, 5′ untranslated regions, introns, protein coding regions, 3′ untranslated regions, polyadenylation sites, and/or transcriptional terminators), sequences that provide DNA transfer and/or integration functions (i.e., site specific recombinase recognition sites, integrase recognition sites), sequences that provide for selective functions (i.e., antibiotic resistance markers, biosynthetic genes), sequences that provide scoreable marker functions (i.e., reporter genes), sequences that facilitate in vitro or in vivo manipulations of the sequences (i.e., polylinker sequences, site specific recombination sequences, homologous recombination sequences), and sequences that provide replication functions (i.e., bacterial origins of replication, autonomous replication sequences, centromeric sequences).

As used herein, the phrases “self”, “selfing”, or “selfed” refer to the process of self-pollinating a plant.

As used herein, the term “transgene” or “transgenic”, refers to any DNA that has been transformed into a plant cell and that has been integrated into a chromosome that is stably maintained in a host cell. In this context, sources for the DNA include, but are not limited to, DNAs from an organism distinct from the host cell organism, species distinct from the host cell species, varieties of the same species that are either distinct varieties or identical varieties. DNA that has been subjected to any in vitro modification, recombinant DNA, and any combination thereof.

To the extent to which any of the preceding definitions is inconsistent with definitions provided in any patent or non-patent reference incorporated herein by reference, any patent or non-patent reference cited herein, or in any patent or non-patent reference found elsewhere, it is understood that the preceding definitions will be used herein.

In certain embodiments, a recessive mutation in a plant's MSH1 gene occurs in a genetically homozygous state to initiate epigenetic modifications (see U.S. patent application Ser. No. 13/462,216, which is incorporated herein by reference in its entirety). In order to restore one or more of the MSH1 gene to their normal non-mutant state, a cross to a line with a normal MSH1 gene is required, followed by selfing or backcrossing to identify progeny with useful traits and containing one or more normal MSH1 genes. Double haploid methods can be applied to the F1 plant or its subsequent selfed or backcrossed generations (F2-Fn), preferably the F2, F3, F4, F5. or F6 generations to provide epigenetically homozygous lines that exhibit improved epigenetic stability and contain non-mutant MSH1 genes.

In certain embodiments, a recessive mutation in a plant's MSH1 gene occurs in a genetically homozygous state to initiate epigenetic modifications (see U.S. patent application Ser. No. 13/462,216, which is incorporated herein by reference in its entirety). In order to restore one or more of the MSH1 gene to their normal non-mutant state, a cross to a genetically different line with a normal MSH1 gene is required, to produce a genetically and epigenetically heterogenous F1 progeny. Said progeny is selfed or backcrossed and screened to identify useful traits in F2 progeny containing one or more normal MSH1 genes. Double haploid methods can be applied to the F1 plant or its subsequent selfed or backcrossed generations (F2-Fn), preferably the F2, F3, F4, F5, or F6 generations to provide epigenetically homozygous lines that exhibit improved epigenetic stability and contain non-mutant MSH1 genes.

In certain embodiments, suppression of a plant's MSH1 gene initiates epigenetic modifications to produce useful traits (see U.S. patent application Ser. No. 13/462,216, U.S. Provisional 61/863,267, U.S. Provisional 61/882,140, and U.S. Provisional 61/901,349. each of which is incorporated herein by reference in its entirety). In order to restore MSH1 function, a plant heterozygous for the suppressing gene can be selfed or backcrossed or outcrossed to identify progeny that are not suppressed for MSH1 function. Double haploid methods can be applied to progeny of said plant or its subsequent selfed, backcrossed, or outcrossed generations (S1-Sn, F2-Fn or the equivalent outcross or backcross generation), preferably the S1-S6, F1-F6, or equivalent generations if outcrossed or backcrossed, to provide epigenetically homozygous lines that exhibit useful traits, improved epigenetic stability, and lack the suppressing gene.

A benefit of utilizing the double haploid method is a rapid method for achieving epigenetically homozygous materials. Most importantly, the rapid achievement of epigenetic homozygosity stabilizes the epigenetic modifications, facilitating the subsequent stable propagation of the epigenetically homozygous line, and thereby provides much more stable epigenetically-derived agronomic traits such as yield increases and stress tolerance in a self-pollinated plant or in a hybrid plant.

In certain embodiments, a parental plant that has been previously suppressed for MSH1 expression to generate epigenetic diversity, is converted to a double haploid plant. The resulting double haploid plant has the same epigenetic modifications on both sets of chromosomes as a consequence of the double haploid process deriving both sets of chromosomes from the haploid stage of the plant. The parental plant can be either genetically homozygous or heterozygous in this embodiment. Methods of suppressing MSH1 function to produce useful epigenetic modifications are described in U.S. patent application Ser. No. 13/462,216, in U.S. Provisional 61/863,267, in U.S. Provisional 61/882,140, and in U.S. Provisional 61/901,349, each of which is incorporated herein by reference in its entirety. In certain embodiments, said parental plant can be genetically homozygous at the level of DNA sequence of the chromosomes. In certain embodiments, said parental plant can be genetically heterozygous at the level of DNA sequence of the chromosomes. In certain embodiments, said parental plant can suppressed for MSH1 by a trans-dominant method such as RNAi or a dominant negative MSH1 mutation, and a resulting progeny lacking any transgene can be derived by selfing the parental plant. Haploid plants can be generated during the selfing step or in subsequent progeny generations. The haploid plant chromosome number is doubled spontaneously or preferably by a colchicine or equivalent chemical treatment to produce a double haploid plant.

In certain embodiments, a parental plant can be suppressed for MSH1 by a recessive method such as such as a weak or null mutations of MSH1. Progeny lacking any mutant MSH11 can be derived by first outcrossing said parental plant to a genetically isogenic or genetically different second plant containing a non-mutant MSH1 gene. The resulting progeny plants can then be selfed or outcrossed or backcrossed to obtain plants lacking a mutation in MSH1 but containing useful epigenetic modifications. Haploid or double haploid plants can be generated during the selfing or outcrossing or backcrossing step or in subsequent progeny generations. The haploid plant chromosome number is doubled spontaneously or by a colchicine treatment or similar doubling method to produce a double haploid plant.

In certain embodiments, an outcross of an individual line or lines exhibiting, containing, or harboring the useful traits can be to a plant or plants that have not been subjected to MSH1 suppression but are otherwise isogenic to the individual line or lines. In certain exemplary embodiments, a line or lines exhibiting, containing, or harboring the useful traits is obtained by suppressing MSH1 function in a given germplasm and can be outcrossed to a plant having that same germplasm that was not subjected to MSH1 suppression. In other embodiments, an outcross of an individual line or lines exhibiting, containing, or harboring the useful traits can be to a plant or plants that have not been subjected to MSH1 suppression but are not isogenic to the individual line(s). Thus, in certain embodiments, an outcross of an individual line or lines exhibiting, containing, or harboring the useful traits can also be to a plant or plants that comprise one or more chromosomal polymorphisms that do not occur in the individual line(s), to a plant or plants derived from partially or wholly different germplasm, or to a plant or plant of a different heterotic group (in instances where such distinct heterotic groups exist). It is also recognized that such an outcross can be made in either direction. Thus, an individual line exhibiting or harboring useful traits can be used as either a pollen donor or a pollen recipient to a plant that has not been subjected to MSH1 suppression in such outcrosses. In certain embodiments, the progeny of the outcross are then selfed or converted to haploid or double haploid plants to establish individual lines that can be separately screened to identify lines with improved traits or harboring improved traits relative to parental lines. Such individual lines that exhibit the improved traits are then selected and can be propagated by further selfing.

In certain embodiments, an outcross of an individual line or lines exhibiting, containing, or harboring the useful traits can be to a plant or plants that have been subjected to MSH1 suppression but are otherwise isogenic to the individual line or lines. In certain exemplary embodiments, a line or lines exhibiting, containing, or harboring the useful traits is obtained by suppressing MSH1 function in a given germplasm and can be outcrossed to a plant having that same germplasm that was subjected to MSH1 suppression. In other embodiments, an outcross of an individual line or lines exhibiting, containing, or harboring the useful traits can be to a plant or plants that have been subjected to MSH1 suppression but are not isogenic to the individual line(s). Thus, in certain embodiments, an outcross of an individual line or lines exhibiting, containing, or harboring the useful traits can also be to a plant or plants that comprise one or more chromosomal polymorphisms that do not occur in the individual line(s), to a plant or plants derived from partially or wholly different germplasm, or to a plant or plant of a different heterotic group (in instances where such distinct heterotic groups exist). It is also recognized that such an outcross can be made in either direction. Thus, an individual line exhibiting or harboring useful traits can be used as either a pollen donor or a pollen recipient to a plant that has been subjected to MSH1 suppression in such outcrosses. In certain embodiments, the progeny of the outcross are then selfed or converted to haploid or double haploid plants to establish individual lines that can be separately screened to identify lines with improved traits or harboring improved traits relative to parental lines. Such individual lines that exhibit the improved traits are then selected and can be propagated by further selfing.

In general, changes in DNA methylation are often accompanied by changes in small RNA (sRNA) profiles, particularly sRNAs of 20 to 24 nucleotides in length and microRNAs (Bond et. al., Trends Cell Biol. 2014 Feb. 24(2):100-7; Bologna et al., Annu Rev Plant Biol. 2014 Feb. 26; Hu et al., Biochem Biophys Res Commun. 2014 Feb. 21; 444(4):676-81.), making assaying sRNA levels an alternative or complementary method for measuring changes in DNA methylation levels. Accordingly, an objective is to identify differences in one or more sRNAs derived from certain altered chromosomal loci between candidate plants and isogenic reference plants not derived from MSH1 suppressed plants. Altered chromosomal loci thus identified can then be isolated or selected in plants to obtain plants useful for plant breeding to develop improved traits selected from the group consisting of improved yield, delayed flowering, non-flowering, increased biotic stress resistance, increased abiotic stress resistance, enhanced lodging resistance, enhanced growth rate, enhanced biomass, enhanced tillering, enhanced branching, delayed flowering time, and delayed senescence.

In certain embodiments, altered chromosomal loci can be identified by identifying sRNAs that are up or down regulated in the candidate plants in comparison to reference plants. These methods are based in part on identification of altered chromosomal loci where small interfering sRNAs direct the methylation of specific gene targets by RNA-directed DNA methylation (RdDM). The RNA-directed DNA methylation (RdDM) process has been described (Chinnusamy V et al. Sci China Ser C-Life Sd. (2009) 52(4): 33 1-343; Bond et. al., Trends Cell Biol. 2014 Feb. 24(2): 100-7). Any applicable technology platform can be used to compare small RNAs in the test and reference plants, including, but not limited to: microarray-based methods (Franco-Zorilla et al. Plant J. 200959(5):840-50); deep sequencing based methods (Wang et al. The Plant Cell 21:1053-1069(2009); Wei et al., Proc Natl Acad Sci USA. 2014 Feb. 19, 111(10): 3877-3882; Zhai et al., Methods. 2013 Jun. 28. pii: S1046-2023(13)00237-5. doi: 10.1016/j.ymeth.2013.06.025 or J. Zhai et al., Methods (2013), http://dx.doi.org/10.1016/j.ymeth.2013.06.025); U.S. Pat. No. 7,550,583: U.S. Pat. No. 8,399,221; U.S. Pat. No. 8,399,222; U.S. Pat. No. 8,404,439; U.S. Pat. No. 8,637,276; Rosas-Cárdenas et al., (2011) Plant Methods 2011, 7:4; Moyano et al., BMC Genomics. 2013 Oct. 11; 14:701; Eldem et al., PLoS One. 2012; 7(12):e50298; Barber et al., Proc Natl Acad Sci USA. 2012 Jun. 26; 109(26):10444-9; Gommans et al., Methods Mol Biol. 2012; 786:167-78; and the like.

DNA methylation and sRNAs corresponding to these regions can change in progeny plants when two parent plants are crossed. Tomato progeny plants from a cross displayed transgressive sRNAs that were more abundant in the progeny than in either parent (Shivaprasad et al., EMBO J. 2012 Jan. 18; 31(2):257-66). A cross between two maize lines, B73 and Mo17, yielded paramutation type switches of the DNA methylation pattern of one parent chromosome being switched to that of the other parental chromosome at the corresponding loci (Regulski et al., Genome Res. 2013 October; 23(10): 1651-62). A cross between Arabidopsis plants produced progeny wherein the DNA methylation patterns of one parental chromosome were imposed onto the other parental chromosome, either gaining or losing DNA methylation levels (Greaves et al., Proc Natl Acad Sci USA. 2014 Feb. 4; 111(5):2017-22). These non-limiting examples indicate DNA methylation patterns can be more complex than just additive patterns from both parents. Accordingly, an objective is to identify new combinations of altered chromosomal loci in progeny plants that have new patterns of DNA methylation and/or of sRNA profiles. New combinations of altered chromosomal loci can result both from segregation of altered chromosomal loci in the progeny as well as due to changes in DNA methylation and sRNA profiles due to transgressive, paramutation type switching, and other biological processes. In certain embodiments, altered chromosomal loci are derived from a parental plant subjected to suppression of MSH1. In certain embodiments, altered chromosomal loci are derived from the formation of new patterns of DNA methylation and sRNA levels from the interaction of altered chromosomal loci derived from a parental plant subjected to suppression of MSH1 with chromosomal loci from a second plant. Said second plant can be from a parental plant subjected to suppression of MSH1 or from a parental plant not subjected to suppression of MSH1. Crossing parental lines both previously subjected to MSH1 suppression and containing different groupings of altered chromosomal loci provides a method of creating new combinations of altered chromosomal loci.

In certain embodiments, altered chromosomal loci can be identified by identifying chromosomal regions (genomic DNA) that have an altered methylation status in the test plants (in comparison to a reference plant). An altered methylation status can comprise either the presence or absence of methylation in one or more chromosomal loci of a test plant in comparison to a reference plant. Any applicable technology can be used to compare the methylation status of chromosomal loci in the test and reference plants. Applicable technologies for identifying chromosomal loci with changes in their methylation status include, but not limited to, methods based on immunoprecipitation of DNA with antibodies that recognize 5-methyl-cytidine, methods based on use of methylation dependent restriction endonucleases and PCR such as McrBC-PCR methods (Rabinowicz, et al. Genome Res. 13: 2658-2664 2003; Li et al., Plant Cell 20:259-276, 2008), sequencing of bisulfite-converted DNA (Frommer et al. Proc. Natl. Acad. Sci. U.S.A. 89 (5): 1827-31; Tost et al. BioTechniques 35 (1): 152-156, 2003), methylation-pericentromeric regions specific PCR analysis of bisulfite treated DNA (Herman et al. Proc. Natl. Acad. Sci. U.S.A. 93 (18): 9821-6, 1996), deep sequencing based methods (Wang et al. The Plant Cell 21:1053-1069 (2009)), methylation sensitive single nucleotide primer extension (MsSnuPE; Gonzalgo and Jones Nucleic Acids Res. 25 (12): 2529-2531, 1997), fluorescence correlation spectroscopy (Umezu et al. Anal Biochem. 415(2): 145-50, 2011), single molecule real time sequencing methods (Flusberg et al. Nature Methods 7, 461-465), high resolution melting analysis (Wojdacz and Dobrovic (2007) Nucleic Acids Res. 35 (6): e41), and the like.

Additional applicable technologies for identifying chromosomal loci with changes in their DNA methylation status include, but not limited to, the preparation, amplification and analysis of Methylome libraries as described in U.S. Pat. No. 8,440,404; using Methylation-specific binding proteins as described in U.S. Pat. No. 8,394,585; determining the average DNA methylation density of a locus of interest within a population of DNA fragments as described in U.S. Pat. No. 8,361,719; by methylation-sensitive single nucleotide primer extension (Ms-SNuPE), for determination of strand-specific methylation status at cytosine residues as described in U.S. Pat. No. 7,037,650; a method for detecting a methylated CpG-containing nucleic acid present in a specimen by contacting the specimen with an agent that modifies unmethylated cytosine and amplifying the CpG-containing nucleic acid using CpG-specific oligonucleotide primers as described in U.S. Pat. No. 6,265,171; an improved method for the bisulfite conversion of DNA for subsequent analysis of DNA methylation as described in U.S. Pat. No. 8,586,302; for treating genomic DNA samples with sodium bisulfite to create methylation-dependent sequence differences, followed by detection with fluorescence-based quantitative PCR techniques as described in U.S. Pat. No. 8,323,890; a method for retaining methylation pattern in globally amplified DNA as described in U.S. Pat. No. 7,820,385; a method for detecting cytosine methylations DNA as described in U.S. Pat. No. 8,241,855; a method for quantification of methylated DNA as described in U.S. Pat. No. 7,972,784; a highly sensitive method for the detection of cytosine methylation patterns as described in U.S. Pat. No. 7,229,759; additional methods for detecting DNA methylation changes are described in U.S. Pat. No. 7,943,308 and U.S. Pat. No. 8,273,528.

Plant centromeres are responsible for normal chromosomal segregation during mitosis and meiosis. Flanking the centromeres are the pericentromeric regions which facilitate centromere function. Centromeres are primarily composed of centromeric satellite repeated sequences and centromeric retrotransposons. In Arabidopsisi, a 180-bp satellite repeat forms the main repeating centromeric sequence. Centromeric satellite repeats are mostly specific to the centromeric regions with a few copies that generally are not present as long tandem repeats elsewhere in the genome. An exception is that a limited amount of centromeric satellite repeats can also be found in the flanking pericentromeric regions. Centromeric regions bind the specialized centromeric histones such as CENH3 and the like.

Accordingly, a functional description of pericentromeric regions is heterochromatic regions containing abundant repeated sequences, transposable elements, and retrotransposons that physically flank the centromeric regions. Pericentromeric regions are often rich in mono and dimethylated H3K9 heterochromatin regions and can contain active genes. At the sequence level, a functional definition for pericentromeric sequences are repeated sequences other than the centromeric repeats and that contain transposable elements and retrotransposons embedded in said repeated pericentromeric sequences. When available, chromosomal positioning information about the location of sequences that are located adjacent to the centromere strengthens the identification ofpericentromeric sequences.

Transposable elements of both class I (long terminal repeat [LTR]-retrotransposons) and class II (DNA transposons of different superfamilies) are abundantly present in plant genomes (Kidwell 2002 Genetica 115:49-63; Kapitonov and Jurka Nat Rev Genet. 2008 May; 9(5):411-2; Wicker Nat Rev Genet. 2007 December; 8(12):973-82). They can be identified by various software programs as described in Lerat (Heredity (Edinb). 2010 June; 104(6):520-33). Repbase Update (RU) is a database of prototypic sequences representing repetitive DNA including transposable elements from different eukaryotic species. Candidate sequences available from a variety of assay methods such as microarrays and next generation sequencing such as Illumina can be compared to known transposable element sequences as described above to identify most known transposable elements in a plant genome.

The aforementioned methods are useful for producing plants with new combinations of altered chromosomal loci and/or identifying plants with useful combinations of altered chromosomal loci. These plants can be further bred and/or screened and selected for useful traits in a manner consistent with plant breeding practices. In certain embodiments, the screened and selected trait is improved plant yield. In certain embodiments, such yield improvements are improvements in the yield of a plant line relative to one or more parental line(s) under non-stress conditions. Non-stress conditions comprise conditions where water, temperature, nutrients, minerals, and light fall within typical ranges for cultivation of the plant species. Such typical ranges for cultivation comprise amounts or values of water, temperature, nutrients, minerals, and light that are neither insufficient nor excessive. In certain embodiments, such yield improvements are improvements in the yield of a plant line relative to parental line(s) under abiotic stress conditions. Such abiotic stress conditions include, but are not limited to, conditions where water, temperature, nutrients, minerals, and/or light that are either insufficient or excessive. Abiotic stress conditions would thus include, but are not limited to, drought stress, osmotic stress, nitrogen stress, phosphorous stress, mineral stress, heat stress, cold stress, and/or light stress. In this context, mineral stress includes, but is not limited to, stress due to insufficient or excessive potassium, calcium, magnesium, iron, manganese, copper, zinc, boron, aluminum, or silicon. In this context, mineral stress includes, but is not limited to, stress due to excessive amounts of heavy metals including, but not limited to, cadmium, copper, nickel, zinc, lead, and chromium.

Improvements in yield in plant lines obtained can be identified by direct measurements of wet or dry biomass including, but not limited to, grain, lint, leaves, stems, or seed. Improvements in yield can also be assessed by measuring yield related traits that include, but are not limited to, 100 seed weight, a harvest index, and seed weight. In certain embodiments, such yield improvements are improvements in the yield of a plant line relative to one or more parental line(s) and can be readily determined by growing plant lines obtained by the methods provided herein in parallel with the parental plants. In certain embodiments, field trials to determine differences in yield whereby plots of test and control plants are replicated, randomized, and controlled for variation can be employed (Giesbrecht F G and Gumpertz M L. 2004. Planning, Construction, and Statistical Analysis of Comparative Experiments. Wiley. New York; Mead, R. 1997. Design of plant breeding trials. In Statistical Methods for Plant Variety Evaluation. eds. Kempton and Fox. Chapman and Hall. London.). Other useful traits that can be obtained by the methods provided herein include various seed quality traits including, but not limited to, improvements in either the compositions or amounts of oil, protein, or starch in the seed. Still other useful traits that can be obtained by methods provided herein include, but are not limited to, increased biomass, non-flowering, male sterility, digestability, seed filling period, maturity (either earlier or later as desired), reduced lodging, and plant height (either increased or decreased as desired).

### Examples

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Epigenetically Enhanced Double Haploid Plants

A crop plant is subjected to a method of suppressing MSH1 function, either a trans-acting silencing method or a loss of function mutation within one or both MSH1 genes to produce useful epigenetic modifications as described herein and in U.S. patent application Ser. No. 13/462,216, in U.S. Provisional 61/863,267, in U.S. Provisional 61/882,140, and in U.S. Provisional 61/901,349, each of which is incorporated herein by reference in its entirety. A loss of function mutation within a MSH1 gene, preferably a stop codon within the 5′ portion of MSH1, is produced and identified by the TILLING method (Henikoff et al., Plant Physiol. 2004, 135:630-636). The resulting MSH1 suppressed plant, its progeny, or its progeny now containing one or more functional MSH1 genes, said plant or progeny either genetically homozygous or heterozygous with respect to its chromosomes derived from its parents, are useful for plant breeding, and/or are selected for useful traits, and/or comprise altered chromosomal loci, and are converted to a double haploid plant by the methods known to those skilled in the art for making haploid and double haploid plants. The haploid and/or double haploid tissue or plant is formed from either the male or female reproductive tissues or cells, thereby inheriting only the male or female chromosomes, respectively. The double haploid plant has the same epigenetic modifications on both sets of chromosomes as a consequence of the double haploid process deriving both sets of chromosomes from the haploid plant or cells exclusively from either the male or female parent, thereby enhancing the double haploid plant's and its progeny's epigenetic stability and useful traits.

### Example 2

### Epigenetically Enhanced CenH3 Method for Generating Double Haploid Plants

A crop plant is subjected to a method of suppressing MSH1 function, either a trans-acting silencing method or a loss of function mutation within one or both MSH1 genes to produce useful epigenetic modifications as described herein and in U.S. patent application Ser. No. 13/462,216, in U.S. Provisional 61/863,267, in U.S. Provisional 61/882,140, and in U.S. Provisional 61/901,349, each of which is incorporated herein by reference in its entirety. A loss of function mutation within a MSH1 gene, preferably a stop codon within the 5′ portion of MSH1, is produced and identified by the TILLING method (Henikoff et al., Plant Physiol. 2004, 135:630-636). The resulting MSH1 suppressed plant, its progeny, or its progeny now containing one or more functional MSH1 genes, said plant or progeny either genetically homozygous or heterozygous with respect to its chromosomes derived from its parents, are useful for plant breeding, and/or are selected for useful traits, and/or comprise altered chromosomal loci, and are converted to a double haploid plant by the methods described in U.S. Pat. No. 8,618,354, incorporated herein by reference in its entirety, which describes CenH3 sequences of many plant species and a method wherein alteration of the plant's CenH3 leads to haploid plants, that can then be colchicine treated to form double haploids. A RNAi construct, of the hairpin design described in U.S. patent application Ser. No. 13/462,216, with homology against the plant's CenH3 mRNA coding sequences, is made to suppress endogenous CenH3. A tailswap synthetic transgene that lacks strong homology (less than 90%, less than 80%, or preferably less than 70% across the entire gene including individual 21 bp regions) to the endogenous CenH3 genes is synthesized commercially and is operably linked and expressed from the Cauliflower Mosaic virus 35S promoter and Nopaline Synthase 3′ polyadenylation region. A DNA vector containing both the CenH3 RNAi gene and the synthetic tailswap CenH3 gene is added to an Agrobacterium T-DNA binary vector by standard recombinant DNA methods and transformed into the target plant tissues by plant transformation methods known to those skilled in the art. Crossing plants that lack an endogenous CenH3 protein and express an active mutated tailswap CenH3 protein as described herein, either as a pollen or ovule parent to a plant that expresses an endogenous CenH3 protein will result in at least some progeny (e.g., at least 0.1%, 0.5%, 1%, 5%, 10%, 20% or more) that are haploid and comprise only chromosomes from the parent plant that expresses the native endogenous CenH3 protein. Double haploid plants are produced by treating the haploid plants or tissues with colchicine and screening for double haploids as described in Wan, et al., “Efficient Production of Doubled Haploid Plants Through Colchicine Treatment of Anther-Derived Maize Callus.” Theoretical and Applied Genetics, 77:889-892 (1989). The resulting double haploid plant has the same epigenetic modifications on both sets of chromosomes as a consequence of the double haploid process deriving both sets of chromosomes from the haploid stage of the method.

### Example 3

### Epigenetically Enhanced Double Haploids of Alfalfa

An alfalfa (Medicago sativa L) plant is subjected to a method of suppressing MSH1 function, either a trans-acting silencing method or a loss of function mutation within one or both MSH1 genes to produce useful epigenetic modifications as described herein and in U.S. patent application Ser. No. 13/462,216, in U.S. Provisional 61/863,267, in U.S. Provisional 61/882,140, and in U.S. Provisional 61/901,349, each of which is incorporated herein by reference in its entirety. A loss of function mutation within a MSH1 gene, preferably a stop codon within the 5′ portion of MSH1, is produced and identified by the TILLING method (Henikoff et al., Plant Physiol. 2004, 135:630-636). Tetraploid alfalfa has 2 pairs of MSH1 genes, requiring TILLING mutations in each of the two homeologous genes, crossing the separate lines to construct an alfalfa line with mutations in both pairs of its MSH1 genes to create a homozygous recessive line suppressed for MSH1 function. Alternatively, the alfalfa MSH1 sequence is obtained by PCR of cDNA from mRNA alfalfa plants, using the closely related Medicago truncutula MSH1 gene (Genbank XM_003590135) as a starting point for designing primers to isolate the alfalfa MSH1 cDNA. Alfalfa transgenic plants expressing a hairpin alfalfa MSH1 RNAi binary Agrobacterium construct according to the design described in in U.S. patent application Ser. No. 13/462,216, are produced by the transformation method described in U.S. Pat. No. 7,521,600, which is hereby incorporated by reference in its entirety. The resulting MSH1 suppressed plant, its progeny, or its progeny now containing one or more functional MSH1 genes, said plant or progeny either genetically homozygous or heterozygous with respect to its chromosomes derived from its parents, are useful for plant breeding, and/or are selected for useful traits, and/or comprise altered chromosomal loci, and are converted to a double haploid plant by the methods known to those skilled in the art for making haploid and double haploid plants. Alfalfa is tetraploid (4n=32), so the haploids (2n=16) are produced by the method of Zagorska and Dimitrov (Plant Cell Rep. 1995 January; 14(4):249-52. doi: 10.1007/BF00233643. Double haploids are produced by the doubling of a set of chromosomes from a haploid plant to produce a diploid plant by colchicine [Wan, et al., “Efficient Production of Doubled Haploid Plants Through Colchicine Treatment of Anther-Derived Maize Callus,” Theoretical and Applied Genetics, 77:889-892 (1989)]. Double haploids alfalfa plants obtained are further bred as described in U.S. Provisional 61/863,267, in U.S. Provisional 61/882,140, and in U.S. Provisional 61/901,349, each of which is incorporated herein by reference in its entirety. The resulting plants can be used as inbreds, or as isogenic hybrids (as described in in U.S. patent application Ser. No. 13/462,216, which is incorporated herein by reference in its entirety), or hybrids wherein the other parent is known to those skilled in the art to be heterotic with the parent of the double haploid parent.

### Example 4

### Epigenetically Enhanced Double Haploids of Canola and Rapeseed

Canola and rapeseed plants (Brassica napus L.) are subjected to a method of suppressing MSH1 function, either a trans-acting silencing method or a loss of function mutation within one or both MSH1 genes to produce useful epigenetic modifications as described herein and in U.S. patent application Ser. No. 13/462,216, in U.S. Provisional 61/863,267, in U.S. Provisional 61/882,140, and in U.S. Provisional 61/901,349, each of which is incorporated herein by reference in its entirety. A loss of function mutation within a MSH1 gene, preferably a stop codon within the 5′ portion of MSH1, is produced and identified by the TILLING method (Henikoff et al., Plant Physiol. 2004, 135:630-636). Alternatively, the Canola MSH1 sequence is obtained by PCR of cDNA from mRNA Canola plants, using the closely related Brassica rapa MSH1 cDNA (Brassica Locus Bra015033 described in U.S. patent application Ser. No. 13/462,216) as a starting point for designing primers to isolate the Canola MSH1 cDNA. Canola and rapeseed transgenic plants expressing a hairpin Canola or rapeseed MSH1 RNAi binary Agrobacterium construct according to the design described in in U.S. patent application Ser. No. 13/462,216, are produced by the transformation method described in U.S. Pat. No. 5,750,871, which is hereby incorporated by reference in its entirety. The resulting MSH1 suppressed plant, its progeny, or its progeny containing one or more functional MSH1 genes, said plant or progeny either genetically homozygous or heterozygous with respect to its chromosomes derived from its parents, are useful for plant breeding, and/or are selected for useful traits, and/or comprise altered chromosomal loci, and are converted to a double haploid plant by the methods known to those skilled in the art for making haploid and double haploid plants. Double haploid canola and rapeseed plants are made by the method of U.S. Pat. No. 6,200,808, which is hereby incorporated by reference in its entirety. The double haploids canola or rapeseed plants obtained are further bred as described in U.S. Provisional 61/863,267, in U.S. Provisional 61/882,140, and in U.S. Provisional 61/901,349, each of which is incorporated herein by reference in its entirety. The resulting plants can be used as inbreds, or as isogenic hybrids (as described in in U.S. patent application Ser. No. 13/462,216, which is incorporated herein by reference in its entirety), or hybrids wherein the other parent is known to those skilled in the art to be heterotic with the parent of the double haploid parent.

### Example 5

### Epigenetically Enhanced Double Haploid Cotton

Cotton plants are subjected to a method of suppressing MSH1 function, either a trans-acting silencing method or a loss of function mutation within one or both MSH1 genes to produce useful epigenetic modifications as described herein and in U.S. patent application Ser. No. 13/462,216, in U.S. Provisional 61/863,267, in U.S. Provisional 61/882,140, and in U.S. Provisional 61/901,349, each of which is incorporated herein by reference in its entirety. A loss of function mutation within a MSH1 gene, preferably a stop codon within the 5′ portion of MSH1, is produced and identified by the TILLING method (Henikoff et al., Plant Physiol. 2004, 135:630-636). Alternatively, for trans-acting silencing, cotton transgenic plants expressing a hairpin cotton MSH1 RNAi binary Agrobacterium construct according to the design described in in U.S. patent application Ser. No. 13/462,216, are produced by the cotton transformation method described in U.S. Pat. No. 5,846,797, which is hereby incorporated by reference in its entirety. The resulting MSH1 suppressed plant, its progeny, or its progeny containing one or more functional MSH1 genes, said plant or progeny either genetically homozygous or heterozygous with respect to its chromosomes derived from its parents, are useful for plant breeding, and/or are selected for useful traits, and/or comprise altered chromosomal loci, and are converted to a double haploid plant by the methods known to those skilled in the art for making haploid and double haploid plants. Double haploid cotton plants are made by crossing to G. barbadense L., using the VSg-7 line of G. barbadense as the female parent, and haploid plants were doubled with an 0.5% aqueous solution of colchicine (Chaudhari Bulletin of the Torrey Botanical Club Vol 106 pp 123-130, 1979). The double haploid cotton plants obtained are further bred as described in U.S. Provisional 61/863,267, in U.S. Provisional 61/882,140, and in U.S. Provisional 61/901,349, each of which is incorporated herein by reference in its entirety. The resulting plants can be used as inbreds, or as isogenic hybrids (as described in in U.S. patent application Ser. No. 13/462,216, which is incorporated herein by reference in its entirety), or hybrids wherein the other parent is known to those skilled in the art to be heterotic with the parent of the double haploid parent.

### Example 6

### Epigenetically Enhanced Double Haploid Corn

Corn plants are subjected to a method of suppressing MSH1 function, either a trans-acting silencing method or a loss of function mutation within one or both MSH1 genes to produce useful epigenetic modifications as described herein and in U.S. patent application Ser. No. 13/462,216, in U.S. Provisional 61/863,267, in U.S. Provisional 61/882,140, and in U.S. Provisional 61/901,349, each of which is incorporated herein by reference in its entirety. A loss of function mutation within a MSH1 gene, preferably a stop codon within the 5′ portion of MSH1, is produced and identified by the TILLING method (Henikoff et al., Plant Physiol. 2004, 135:630-636). Alternatively, for trans-acting silencing, corn transgenic plants expressing a hairpin corn MSH1 RNAi binary Agrobacterium construct according to the design described in in U.S. patent application Ser. No. 13/462,216, or with an additional monocot intron for increased expression as described in U.S. Provisional 61/901,349 (each of which is incorporated herein by reference in its entirety), are produced by the corn transformation method described in U.S. Pat. No. 7,682,829, which is hereby incorporated by reference in its entirety. The resulting MSH1 suppressed plant, its progeny, or its progeny now containing one or more functional MSH1 genes, said plant or progeny either genetically homozygous or heterozygous with respect to its chromosomes derived from its parents, are useful for plant breeding, and/or are selected for useful traits, and/or comprise altered chromosomal loci, and are converted to a double haploid plant by the methods known to those skilled in the art for making haploid and double haploid plants. Double haploid corn plants are made the method U.S. Pat. No. 5,322,789, incorporated by reference in its entirety. The double haploid corn plants obtained are further bred as described in U.S. Provisional 61/863,267, in U.S. Provisional 61/882,140, and in U.S. Provisional 61/901,349, each of which is incorporated herein by reference in its entirety. The resulting plants can be used as inbreds, or as isogenic hybrids (as described in in U.S. patent application Ser. No. 13/462,216, which is incorporated herein by reference in its entirety), or hybrids wherein the other parent is known to those skilled in the art to be heterotic with the parent of the double haploid parent.

### Example 7

### Epigenetically Enhanced Double Haploid Rice

Rice plants are subjected to a method of suppressing MSH1 function, either a trans-acting silencing method or a loss of function mutation within one or both MSH1 genes to produce useful epigenetic modifications as described herein and in U.S. patent application Ser. No. 13/462,216, in U.S. Provisional 61/863,267. in U.S. Provisional 61/882,140, and in U.S. Provisional 61/901,349, each of which is incorporated herein by reference in its entirety. A loss of function mutation within a MSH1 gene, preferably a stop codon within the 5′ portion of MSH1, is produced and identified by the TILLING method (Henikoff et al., Plant Physiol. 2004, 135:630-636). Alternatively, for trans-acting silencing, rice transgenic plants expressing a hairpin rice MSH1 RNAi binary Agrobacterium construct according to the design described in in U.S. patent application Ser. No. 13/462,216, or with an additional monocot intron for increased expression as described in U.S. Provisional 61/901,349 (each of which is incorporated herein by reference in its entirety). For Indica rice plant, the indica rice transformation method is used as described in U.S. Pat. No. 6,329,571, herein incorporated by reference in its entirety. For japonica rice varieties, the japonica rice transformation method is used as described in U.S. Pat. No. 5,591,616, herein incorporated by reference in its entirety. The resulting MSH1 suppressed plant, its progeny, or its progeny now containing one or more functional MSH1 genes, said plant or progeny either genetically homozygous or heterozygous with respect to its chromosomes derived from its parents, are useful for plant breeding, and/or are selected for useful traits, and/or comprise altered chromosomal loci, and are converted to a double haploid plant by the methods known to those skilled in the art for making haploid and double haploid plants. Double haploid japonica or indica rice plants are made the method of Datta et. al., Plant ScienceVolume 67, Issue 1, 1990, Pages 83-88. The double haploid rice plants obtained are further bred as described in U.S. Provisional 61/863,267, in U.S. Provisional 61/882,140, and in U.S. Provisional 61/901,349, each of which is incorporated herein by reference in its entirety. The resulting plants can be used as inbreds, or as isogenic hybrids (as described in in U.S. patent application Ser. No. 13/462,216, which is incorporated herein by reference in its entirety), or hybrids wherein the other parent is known to those skilled in the art to be heterotic with the parent of the double haploid parent.

### Example 8

### Epigenetically Enhanced Double Haploid Wheat

Wheat plants are subjected to a method of suppressing MSH1 function, either a trans-acting silencing method or a loss of function mutation within one or both MSH1 genes to produce useful epigenetic modifications as described herein and in U.S. patent application Ser. No. 13/462,216, in U.S. Provisional 61/863,267, in U.S. Provisional 61/882,140, and in U.S. Provisional 61/901,349, each of which is incorporated herein by reference in its entirety. A loss of function mutation within a MSH1 gene, preferably a stop codon within the 5′ portion of MSH1, is produced and identified by the TILLING method (Henikoff et al., Plant Physiol. 2004, 135:630-636). Hexaploid wheat has 3 pairs of homeologous MSH1 genes, requiring TILLING mutations in each of the three pairs of homeologous genes, crossing the separate lines to construct a wheat line with mutations in all 3 pairs of its MSH1 genes to create a homozygous recessive line suppressed for MSH1 function. Alternatively, for trans-acting silencing, wheat transgenic plants expressing a hairpin wheat MSH1 RNAi binary Agrobacterium construct according to the design described in in U.S. patent application Ser. No. 13/462,216, or with an additional monocot intron for increased expression as described in U.S. Provisional 61/901,349 (each of which is incorporated herein by reference in its entirety). For transgenic wheat plants, the wheat genotype-independent transformation method is used as described in U.S. Pat. No. 8,212,109, herein incorporated by reference in its entirety. The resulting MSH1 suppressed plant, its progeny, or its progeny now containing one or more functional MSH1 genes, said plant or progeny either genetically homozygous or heterozygous with respect to its chromosomes derived from its parents, are useful for plant breeding, and/or are selected for useful traits, and/or comprise altered chromosomal loci, and are converted to a double haploid plant by the methods known to those skilled in the art for making haploid and double haploid plants. Double haploid wheat plants are made the method of U.S. Pat. No. 6,812,028, herein incorporated by reference in its entirety. The double haploid wheat plants obtained are further bred as described in U.S. Provisional 61/863,267, in U.S. Provisional 61/882,140, and in U.S. Provisional 61/901,349, each of which is incorporated herein by reference in its entirety. The resulting plants can be used as inbreds, or as isogenic hybrids (as described in in U.S. patent application Ser. No. 13/462,216, which is incorporated herein by reference in its entirety), or hybrids wherein the other parent is known to those skilled in the art to be heterotic with the parent of the double haploid parent.

### Example 9

### Epigenetically Enhanced Double Haploid Barley

Barley plants are subjected to a method of suppressing MSH1 function, either a trans-acting silencing method or a loss of function mutation within one or both MSH1 genes to produce useful epigenetic modifications as described herein and in U.S. patent application Ser. No. 13/462,216, in U.S. Provisional 61/863,267, in U.S. Provisional 61/882,140, and in U.S. Provisional 61/901,349, each of which is incorporated herein by reference in its entirety. A loss of function mutation within a MSH1 gene, preferably a stop codon within the 5′ portion of MSH1, is produced and identified by the TILLING method (Henikoff et al., Plant Physiol. 2004, 135:630-636). Alternatively, for trans-acting silencing, barley transgenic plants expressing a hairpin barley (using Genbank AK369942.1 sequence) MSH1 RNAi binary Agrobacterium construct according to the design described in U.S. patent application Ser. No. 13/462,216, or with an additional monocot intron for increased expression as described in U.S. Provisional 61/901,349 (each of which is incorporated herein by reference in its entirety). For transgenic barley plants, the barley transformation method is used as described in U.S. Pat. No. 6,100,447, herein incorporated by reference in its entirety. The resulting MSH1 suppressed plant, its progeny, or its progeny now containing one or more functional MSH1 genes, said plant or progeny either genetically homozygous or heterozygous with respect to its chromosomes derived from its parents, are useful for plant breeding, and/or are selected for useful traits, and/or comprise altered chromosomal loci, and are converted to a double haploid plant by the methods known to those skilled in the art for making haploid and double haploid plants. Double haploid barley plants are made the method of U.S. Pat. No. 6,812,028, herein incorporated by reference in its entirety. The double haploid barley plants obtained are further bred as described in U.S. Provisional 61/863,267, in U.S. Provisional 61/882,140, and in U.S. Provisional 61/901,349, each of which is incorporated herein by reference in its entirety. The resulting plants can be used as inbreds, or as isogenic hybrids (as described in in U.S. patent application Ser. No. 13/462,216, which is incorporated herein by reference in its entirety), or hybrids wherein the other parent is known to those skilled in the art to be heterotic with the parent of the double haploid parent.

### Example 10

### Epigenetically Enhanced Double Haploid Sorghum

Sorghum plants were subjected to a method of suppressing MSH1 function, either a trans-acting silencing method or a loss of function mutation within one or both MSH1 genes to produce useful epigenetic modifications as described herein and in U.S. patent application Ser. No. 13/462,216, in U.S. Provisional 61/863,267, in U.S. Provisional 61/882,140, and in U.S. Provisional 61/901,349, each of which is incorporated herein by reference in its entirety. A loss of function mutation within a MSH1 gene, preferably a stop codon within the 5′ portion of MSH11, is produced and identified by the TILLING method (Henikoff et al., Plant Physiol. 2004, 135:630-636). Alternatively, for trans-acting silencing, epigenetically enhanced sorghum plants were obtained as described in U.S. patent application Ser. No. 13/462,216, herein incorporated by reference in its entirety. The resulting MSH1 suppressed plant, its progeny, or its progeny now containing one or more functional MSH1 genes, said plant or progeny either genetically homozygous or heterozygous with respect to its chromosomes derived from its parents, are useful for plant breeding, and/or are selected for useful traits, and/or comprise altered chromosomal loci, and are converted to a double haploid plant by the methods known to those skilled in the art for making haploid and double haploid plants. Double haploid sorghum plants are made the method of Elkonin et. al., Euphytica 80:111-118, 1994. The double haploid sorghum plants obtained are further bred as described in U.S. Provisional 61/863,267, in U.S. Provisional 61/882,140, and in U.S. Provisional 61/901,349, each of which is incorporated herein by reference in its entirety. The resulting plants can be used as inbreds, or as isogenic hybrids (as described in in U.S. patent application Ser. No. 13/462,216, which is incorporated herein by reference in its entirety), or hybrids wherein the other parent is known to those skilled in the art to be heterotic with the parent of the double haploid parent.

### Example 11

### Epigenetically Enhanced Double Haploid Potato

Potato (Solanum tuberosum) plants are subjected to a method of suppressing MSH1 function, either a trans-acting silencing method or a loss of function mutation within one or both MSH1 genes to produce useful epigenetic modifications as described herein and in U.S. patent application Ser. No. 13/462,216, in U.S. Provisional 61/863,267, in U.S. Provisional 61/882,140, and in U.S. Provisional 61/901,349, each of which is incorporated herein by reference in its entirety. A loss of function mutation within a MSH1 gene, preferably a stop codon within the 5′ portion of MSH1, is produced and identified by the TILLING method (Henikoff et al., Plant Physiol. 2004, 135:630-636). Tetraploid potato has 2 pairs of homeologous MSH11 genes, requiring TILLING mutations in each of the two pairs of homeologous genes, crossing the separate lines to construct a potato line with mutations in both pairs of its MSH11 genes to create a homozygous recessive line suppressed for MSH1 function. Alternatively, for trans-acting silencing, potato transgenic plants expressing a hairpin tomato (as potato and tomato sequences are nearly identical) or a hairpin potato (sequence in Genbank XM_006340822.1) MSH1 RNAi binary Agrobacterium construct according to the design described in in U.S. patent application Ser. No. 13/462,216. For transgenic potato plants, the potato transformation method is used as described in U.S. Pat. No. 7,250,554, herein incorporated by reference in its entirety. The resulting MSH1 suppressed plant, its progeny, or its progeny now containing one or more functional MSH1 genes, said plant or progeny either genetically homozygous or heterozygous with respect to its chromosomes derived from its parents, are useful for plant breeding, and/or are selected for useful traits, and/or comprise altered chromosomal loci, and are converted to a double haploid plant by the methods known to those skilled in the art for making haploid and double haploid plants. Double haploid (4n=48) potato plants are made the method of Peloquin et al., Annals of Botany 77: 539-542, 1996, to produce haploid potatoes (2n=24), which are then treated with colchicine to make double haploid potatoes (4n=48). The double haploid potato plants obtained are propagated vegetatively or further bred as described in U.S. Provisional 61/863,267, in U.S. Provisional 61/882,140, and in U.S. Provisional 61/901,349, each of which is incorporated herein by reference in its entirety. The resulting plants can be used as inbreds, or as isogenic hybrids (as described in in U.S. patent application Ser. No. 13/462,216, which is incorporated herein by reference in its entirety), or hybrids wherein the other parent is known to those skilled in the art to be heterotic with the parent of the double haploid parent.

### Example 12

### Epigenetically Enhanced Double Haploid Tomato

Tomato plants are produced by the method of suppressing MSH1 function, either a trans-acting silencing method or a loss of function mutation within one or both MSH1 genes to produce useful epigenetic modifications as described herein and in U.S. patent application Ser. No. 13/462,216, in U.S. Provisional 61/863,267, in U.S. Provisional 61/882,140, and in U.S. Provisional 61/901,349, each of which is incorporated herein by reference in its entirety. A loss of function mutation within a MSH1 gene, preferably a stop codon within the 5′ portion of MSH1, is produced and identified by the TILLING method (Henikoff et al., Plant Physiol. 2004, 135:630-636). Alternatively, for trans-acting silencing, the method of suppressing MSH1 function was used in tomatoes to produce useful epigenetic modifications as described in U.S. patent application Ser. No. 13/462,216, which is incorporated herein by reference in its entirety. The resulting MSH1 suppressed plant, its progeny, or its progeny now containing one or more functional MSH1 genes, said plant or progeny either genetically homozygous or heterozygous with respect to its chromosomes derived from its parents, are useful for plant breeding, and/or are selected for useful traits, and/or comprise altered chromosomal loci, and are converted to a double haploid plant by the methods known to those skilled in the art for making haploid and double haploid plants. Double haploid tomato plants are made by the method of U.S. Pat. No. 4,835,339, herein incorporated by reference in its entirety. The double haploid tomato plants obtained are propagated vegetatively or further bred as described in U.S. Provisional 61/863,267, in U.S. Provisional 61/882,140, and in U.S. Provisional 61/901,349, each of which is incorporated herein by reference in its entirety. The resulting plants can be used as inbreds, or as isogenic hybrids (as described in in U.S. patent application Ser. No. 13/462,216, which is incorporated herein by reference in its entirety), or hybrids wherein the other parent is known to those skilled in the art to be heterotic with the parent of the double haploid parent.

### Example 13

### Epigenetically Enhanced Double Haploid Sugar Beets

Sugar beet (Beta vulgaris) plants are subjected to a method of suppressing MSH1 function, either a trans-acting silencing method or a loss of function mutation within one or both MSH1 genes to produce useful epigenetic modifications as described herein and in U.S. patent application Ser. No. 13/462,216, in U.S. Provisional 61/863,267, in U.S. Provisional 61/882,140, and in U.S. Provisional 61/901,349, each of which is incorporated herein by reference in its entirety. A loss of function mutation within a MSH1 gene, preferably a stop codon within the 5′ portion of MSH1, is produced and identified by the TILLING method (Henikoff et al., Plant Physiol. 2004, 135:630-636). Alternatively, for trans-acting silencing, the sugar beet MSH1 sequence is obtained by PCR of cDNA from mRNA sugar beet plants or by commercial synthesis, using the sequence from the Beta vulgaris genome sequence (http://bvseq.molgen.mpg.de/: Bvchr9.sca026:2804420..2805145). Sugar beet transgenic plants expressing a hairpin sugar beet MSH1 RNAi binary Agrobacterium construct according to the design described in in U.S. patent application Ser. No. 13/462,216. For transgenic sugar beet plants, the sugar beet transformation method is used as described in U.S. Pat. No. 6,531,649, herein incorporated by reference in its entirety. The resulting MSH1 suppressed plant, its progeny, or its progeny now containing one or more functional MSH1 genes, said plant or progeny either genetically homozygous or heterozygous with respect to its chromosomes derived from its parents, are useful for plant breeding, and/or are selected for useful traits, and/or comprise altered chromosomal loci, and are converted to a double haploid plant by the methods known to those skilled in the art for making haploid and double haploid plants. Double haploid sugar beet plants are made the method of Weich and Levall, “Doubled haploid production of sugar beet (Beta vulgaris L.)” pp 255-263 in Doubled Haploid Production in Crop Plants 2003 Publisher Springer Netherlands., to produce haploid sugar beets, which are then treated with colchicine to make double haploid sugar beets. The double haploid sugar beets plants obtained are further bred as described in U.S. Provisional 61/863,267, in U.S. Provisional 61/882,140, and in U.S. Provisional 61/901,349, each of which is incorporated herein by reference in its entirety. The resulting plants can be used as inbreds, or as isogenic hybrids (as described in in U.S. patent application Ser. No. 13/462,216, which is incorporated herein by reference in its entirety), or hybrids wherein the other parent is known to those skilled in the art to be heterotic with the parent of the double haploid parent.

### Example 14

### Epigenetically Enhanced CenH3 Method for Generating Double Haploid Soybeans

A soybean plant is subjected to a method of suppressing MSH1 function, either a trans-acting silencing method or a loss of function mutation within one or both MSH1 genes to produce useful epigenetic modifications as described herein and in U.S. patent application Ser. No. 13/462,216, in U.S. Provisional 61/863,267, in U.S. Provisional 61/882,140, and in U.S. Provisional 61/901,349, each of which is incorporated herein by reference in its entirety. A loss of function mutation within a MSH1 gene, preferably a stop codon within the 5′ portion of MSH1, is produced and identified by the TILLING method (Henikoff et al., Plant Physiol. 2004, 135:630-636). Tetraploid soybeans have 2 pairs of homeologous MSH1 genes, requiring TILLING mutations in each of the two pairs of homeologous genes, crossing the separate lines to construct a soybean line with mutations in both pairs of its MSH1 genes to create a homozygous recessive line suppressed for MSH1 function. Alternatively, for trans-acting silencing, a RNAi hairpin method was used as described in U.S. patent application Ser. No. 13/462,216, in U.S. Provisional 61/863,267, in U.S. Provisional 61/882,140, and in U.S. Provisional 61/901,349, each of which is incorporated herein by reference in its entirety. The resulting MSH1 suppressed plant, its progeny, or its progeny now containing one or more functional MSH1 genes, said plant or progeny either genetically homozygous or heterozygous with respect to its chromosomes derived from its parents, are useful for plant breeding, and/or are selected for useful traits, and/or comprise altered chromosomal loci, and are converted to a double haploid plant by the methods described in U.S. Pat. No. 8,618,354, incorporated herein by reference in its entirety, as follows.

The soybean CenH3 described in U.S. Pat. No. 8,618,354 is essentially identical to the Genbank mRNA XM_003528751, which has the amino acid sequence of SEQ ID 1.

A BLAST of this soybean XM_003528751 CenH3 mRNA against the soybean genome at GmGDB identified only two genes with significant homology: Glyma07g06310 and Glyma16g02950, indicating these are the only two copies of CenH3 in the soybean genome. Three separate RNAi constructs, of the hairpin construct design described in U.S. patent application Ser. No. 13/462,216, with homology against the soybean's XM_003528751 CenH3 mRNA coding sequences, are constructed from commercially available synthetic DNA. These 3 separate RNAi constructs use hairpin regions to sequences 1-240, 250-600, or 610-960, respectively, of SEQ ID 2 (mRNA XM_003528751). Each of these RNAi suppression constructs are separately added to a vector containing a synthetic tailswap CenH3 gene designed to lack homology to the hairpin regions, resulting in three separate vectors.

A tailswap synthetic soybean CenH3 transgene is designed and commercially synthesized to encode the tailswap soybean CenH3 protein using alternative codon choices to minimize homology to the RNAi hairpin constructs. The tailswap synthetic soybean CenH3 transgene has the Arabidopsis H3 N-terminal tail sequence (SEQ ID 3) attached to the soybean CenH3 histone fold sequence (SEQ ID 4). The combined tailswap synthetic soybean CenH3 protein sequence (SEQ ID 5) and synthetic gene sequence (SEQ ID 6) are indicated below. SEQ ID 6 lacks significant (<90%) homology to the endogenous soybean CenH3 gene (XM_003528751 CenH3 mRNA) due to alternative codon choices

The tailswap synthetic DNA sequence SEQ ID 6 encoding the soybean tailswap protein is commercially synthesized and operably linked and expressed from the Cauliflower Mosaic virus 35S promoter and Nopaline Synthase 3′ polyadenylation region. The resulting DNA vector containing one of the three CenH3 RNAi hairpin genes and the synthetic tailswap CenH3 gene is added to an Agrobacterium T-DNA binary vector by standard recombinant DNA methods and transformed into the target plant tissues by soybean plant transformation methods as described in U.S. Pat. No. 8,592,212, which is incorporated herein by reference in its entirety. Crossing soybean plants that lack an endogenous CenH3 protein and express an active mutated tailswap CenH3 protein as described herein, either as a pollen or ovule parent to a plant that expresses an endogenous CenH3 protein will result in at least some progeny (e.g., at least 0.1%, 0.5%, 1%, 5%, 10%, 20% or more) that are haploid and comprise only chromosomes from the parent plant that expresses the native endogenous CenH3 protein. Double haploid plants are produced by treating the haploid plants or tissues with colchicine and screening for double haploids as described in Wan, et al., “Efficient Production of Doubled Haploid Plants Through Colchicine Treatment of Anther-Derived Maize Callus,” Theoretical and Applied Genetics, 77:889-892 (1989). The resulting double haploid plant has the same epigenetic modifications on both sets of chromosomes as a consequence of the double haploid process deriving both sets of chromosomes from the haploid stage of the method.

The embodiments were chosen and described in order to best explain the principles of the invention and its practical application to thereby enable others skilled in the art to best utilize the invention in various embodiments and with various modifications as are suited to the particular use contemplated.

As various modifications could be made in the constructions and methods herein described and illustrated without departing from the scope of the invention, it is intended that all matter contained in the foregoing description or shown in the accompanying drawings shall be interpreted as illustrative rather than limiting. Thus, the breadth and scope of the present invention should not be limited by any of the above-described exemplary embodiments, but should be defined only in accordance with the following claims appended hereto and their equivalents.

Sample claims of various inventive aspects of the disclosed invention, not to be considered as exhaustive or limiting, all of which are fully described so as to satisfy the written description, enablement, and best mode requirement of the Patent Laws, are as follows:

Sequence listings

SEQ ID 1(amino acid sequence of soybean CenH3 from Genbank mRNA XM_003528751):
MARVKHTPASRKSAKKQAPRASTSTQPPPQSQSPATRERRRAQQVEPQQEPEAQGRKK
RRNRSGTVALREIRHFQRSCELLIPAAPFIRCVKQITNQFSTEVSRWTPEAVVALQEAAEE
YLVHLFEDGMLCAIHARRITLMKKDIELARRLGGIGRPW
SEQ ID 2 (mRNA sequence of soybean CenH3 from Genbank mRNA XM 0035287511):

| | |
|---|---|
| 1 | aaaccgcttc aEtttgaaaa ttcacaaacc aatagtgaaa agagacagag agggaaaaag |
| 61 | aagaagaaaa gaaaaagaaa attgcaaaaa gcgaaaccga aaaccaaatc cgaagagtga |
| 121 | aggttcgttg aatcccatgg cgagagtgaa gcacacgcca gcttctcgca aatccgctaa |
| 181 | aaagcaagcg ccacgcgcat ccacttccac gcagccgcca ccacaatccc aatcgcctgc |
| 241 | aactagagag aggaggagag ctcaacaagt ggagccgcag caggagcctg aggctcaggg |
| 301 | aaggaagaag aggcgcaata ggtcgggaac tgtggcgctt cgtgagaEtc ggcattttca |
| 361 | gaggagttgt gagcttctca tcccggctgc tccctttatc agatgtgtca aacagattac |
| 421 | aaatcaattc tctacggagg tgtctcgtEg gacacctgaa gctgtggtag cacttcagga |
| 481 | ggcagctgag gaatatctgg ttcacttatt tgaagatgga atgctctgtg caattcatgc |
| 541 | aaggcgtatt actcttatga aaaaggacat tgagttggcc cggaggcttg gaggaatagg |
| 601 | aaggccttgg tgagtggacg atactagtag aagaccattg gttcttttgt ttgtaaaagt |
| 661 | tcacacagta gggggatttc aatgaaggga ggcattggtg cttctgtact ccgcactcta |
| 721 | attgcagtga ttatttgata ttaaatgtta ggaactagac actaactaat gtatgccaat |
| 781 | ggttatatct ctgtaggtga aacaaccatt atagaatggt tgctactata actttaaatt |
| 841 | gatgagtgag gtaatagtgt aacattgtag caatgaagta ttctaagatt attgtcattc |
| 901 | atgttgattt tctgtggtag aaggaattca agggtccaca aaatgtcaag gcttaaaatt |
| | ttcc |

SEQ ID 3 Arabidopsis H3 tail sequence):
MARTKQSARKSHGGKAPTKQLATKAARKSAPTTGGVKKPHFR
SEQ ID 4 (Soybean CenH3 histone fold protein sequence):
PGTVALREIRHFQRSCELLIPAAPFIRCVKQITNQFSTEVSRWTPEAVVALQEAAEEYLVH
LFEDGMLCAIHARRTTLMKKDIELARRLGGIGRPW
SEQ ID 5 (soybean tailswap protein containing an Arabidopsis N-terminal
H3 tail and a soybean CenH3 histone fold region):
MARTKQSARKSHGGKAPTKQLATKAARKSAPTTGGVKKPHRFRPGTVALREIRHFQRS
CELLIPAAPFIRCVKQITNQFSTEVSRWTPEAVVALQEAAEEYLVHLFEDGMLCAIHARRI
TLMKKDIELARRLGGIGRPW
SEQ ID 6 (tailswap synthetic DNA sequence encoding the soybean tailswap protein):
ACCACC
ATGGCTCGTACGAAGCAATCCGCGAGAAAATCACACGGAGGAAAAGCTCCGACGA
AGCAG
CTCGCTACCAAGGCGGCAAGGAAATCTGCACCGACTACCGGAGGAGTCAAGAAACC
TcAc
CGTTTCCGTCCGGGCACCGTGGCCCTCCGGGAGATCCGGCACTICCAG
AGGAGCTGCGAGCTCCTGATCCCCGCAGCACCGTTCATCAGGTGCGTGAAGCAGAT
CACC
AACCAGTTCAGCACTGAGGTCAGCCGGTGGACCCCAGAGGCCGTCGTCGCTCTCCA
GGAG
GCGGCAGAGGAGTATCTCGTCCACCTGTTCGAGGACGGCATGCTCTGCGCCATCCAC
GCC
AGGCGCATCACCCTCATGAAGAAGGACATCGAGCTCGCGCGCAGACTCGGAGGCAT
CGGC
AGACCGTGGTGATGA ACACAC

What is claimed is:

1. A method for producing a haploid or double haploid plant or plant cell useful for plant breeding comprising the steps of:
a. crossing or self-pollinating or culturing gametophytic tissue from a plant or plant cell, wherein said plant or plant cell or a progenitor thereof is or had been subjected to MSH1 suppression; and,
b. producing a haploid or double haploid plant or plant cell from the crossing or self-pollination or culturing gametophytic tissue of step (a) or from progeny thereof, thereby producing a haploid or double haploid plant or plant cell useful for plant breeding.

2. The method of claim 1, wherein the haploid or double haploid plant is produced from progeny of the F1, F2, F3, F4, F5, F6 or S1, S2, S3, S4, S5 or S6 generations.

3. The method of claim 1, wherein the method of producing a haploid or double haploid plant utilizes CenH3 suppression and/or a tailswap CenH3 protein.

4. The method of claim 1, wherein the plant is a corn, rice, wheat, sorghum, barley, soybean, cotton, canola, sugarbeet, alfalfa, potato, or tomato plant.

5. The method of claim 1, wherein the crossing of step (a) is between plants that are essentially isogenic.

6. A method for identifying a double haploid plant harboring a useful trait comprising the steps of:
a. crossing a candidate plant to a second plant, wherein the candidate plant is a double haploid plant or its progeny derived from: (i) a selfed plant wherein said selfed plant or a progenitor thereof is or had been subjected to MSH1 suppression; or (ii) a cross wherein at least one parental plant or a progenitor thereof is or had been subjected to MSH1 suppression; and,
b. identifying one or more progeny plants from the cross in step (a) that have a useful trait, to a greater extent than the candidate plant, the second plant, or a control plant, thereby identifying a double haploid plant harboring a useful trait.

7. The method of claim 6, wherein the control plant is progeny of a cross between: (i) a plant that is not progeny of a selfed plant, a crossed plant, or parent thereof that is or had been subjected to MSH1 suppression; and (ii) a plant that is isogenic to the second plant.

8. A plant or progeny thereof that exhibits a useful trait that is made by the method of claim 1.

9. A plant or progeny thereof that exhibits a useful trait that is made by the method of claim 6.

10. A plant part of the plant or progeny of claim 8, wherein said part is selected from the group consisting of a seed, leaf, stem, fruit, and a root.

11. A plant part of the plant or progeny of claim 9, wherein said part is selected from the group consisting of a seed, leaf, stem, fruit, and a root.

12. A processed plant product or clonal propagate obtained from the plant part of claim 10.

13. A processed plant product or clonal propagate obtained from the plant part of claim 11.

14. A method for producing a haploid or double haploid plant or plant cell useful for plant breeding comprising the steps of:
a. Culturing a plant cell, wherein said plant cell is or had been subjected to MSH1 suppression or is derived from a plant or a progenitor plant thereof that is or had been subjected to MSH1 suppression; and,
b. producing a haploid or double haploid plant from the plant cell of step (a) or from progeny thereof, thereby producing a haploid or double haploid plant or plant cell useful for plant breeding.

15. A plant or plant seed, wherein at least one of the progenitor plants is a plant produced by the method of claim 14.

16. A plant part of the plant or progeny of claim 15, wherein said part is selected from the group consisting of a seed, leaf, stem, fruit, and a root.

17. A processed plant product or clonal propagate obtained from the plant part of claim 16.
